(19) United States
(12) Patent Application Publication
(10) Pub. No.: US 2022/0024925 A1
(43) Pub. Date: Jan. 27, 2022

(54) Deuterium-Substituted 7-Substituted-2-(Benzylamino)-6-Ozopurine Compounds and Uses Thereof

(71) Applicant: Acurx Pharmaceuticals LLC, White Plains, NY (US)

(72) Inventor: Xiang Y. YU, Acton, MA (US)

(21) Appl. No.: 17/380,865

(22) Filed: Jul. 20, 2021

Related U.S. Application Data
(60) Provisional application No. 63/055,159, filed on Jul. 22, 2020.

(51) Int. Cl. C07D 473/30; C07B 59/00

(52) U.S. Cl. CPC C07D 473/30; C07B 2200/05; C07B 59/002

(57) ABSTRACT

The invention relates to compounds of formula (I): The compounds are useful as antibacterial agents, especially again Clostridium difficile-associated diseases.

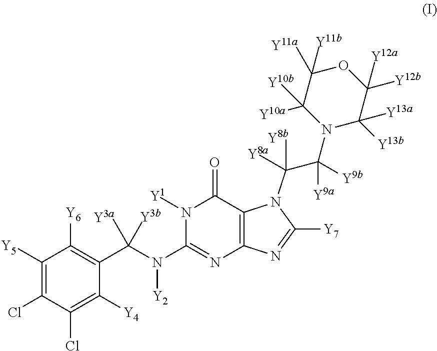

### BACKGROUND OF THE INVENTION

Bacterial pathogens pose a serious threat to public health. Two of the Gram-positive pathogens, Staphylococcus aureus and Enterococcus fecalis/fecium, are primarily nosocomial (hospital-acquired) pathogens; together, they presently account for the majority of nosocomial diseases. Gram-negative bacteria such as Escherichia coli, Salmonella typhimurium, and Pseudomonas aeruginosa, also cause significant diseases in humans. These organisms are aerobic bacteria, i.e., ones that grow in oxygen-containing atmospheres.

Important to health are “anaerobic” bacteria, i.e., those which grow in oxygen-depleted atmospheres, such as are found in intestinal milieu. Gram-positive anaerobes, such as Lactobacilli, Bifidobacteria, and Eubacteria, and Gram-negative anaerobes, such as Bacteroides, represent “good” intestinal organisms important to health, whereas the Gram-positive anaerobes Clostridium difficile and Clostridium perfringens represent pathogenic bacteria. Clostridium difficile (C. diff.) has been increasingly associated with disease in human patients, ironically often as a result of treatment with certain antibiotic drugs.

Clostridioides difficile Infection (CDI) causes ˜476,000 life-threatening gastrointestinal infections leading to ˜25,000 deaths in the US annually, and ˜124,000 health-care associated infections in Europe. Current treatment options are limited and can be associated with recurrent infections and emerging antibacterial resistance. Ibezapolstat, 2-((3,4-Dichlorobenzyl)amino)-7-(2-morpholinoethyl)-1,7-dihydro-6H-purin-6-one, is a novel oral antibiotic with no inherent resistance. Ibezapolstat inhibits DNA polymerase IIIC, an enzyme essential for DNA replication of most Gram-positive pathogens.

### SUMMARY OF THE INVENTION

It has been found that 2-((3,4-dichlorobenzyl)amino)-7-(2-morpholinoethyl)-1,7-dihydro-6H-purin-6-one (hereinafter “Compound I-1”) shown below has potent activity against the growth of the intestinal anaerobe C. diff, but weak activity against other, intestinal Gram-positive anaerobes. The compounds can be administered to reduce the likelihood of developing or to treat C. diff. infections in human patients. See Wright et al., U.S. Pat. No. 8,796,292.

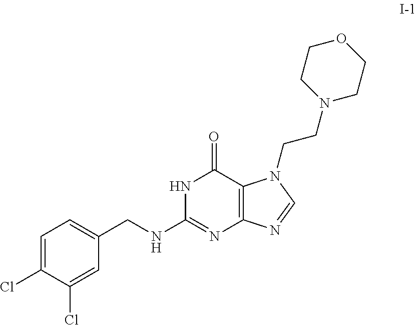 [SMILES: O=c1[nH]c(NCc2ccc(Cl)c(Cl)c2)nc2ncn(CCN3CCOCC3)c12]

Deuterated analogs may improve a compound's properties. For example, some deuterated analogs are known to have a longer half-life when administered to a subject without affecting the compound's bioavailability or safety.

The present invention relates to deuterated analogs of Compound I-1. In one aspect, the invention includes a compound represented by Formula I:

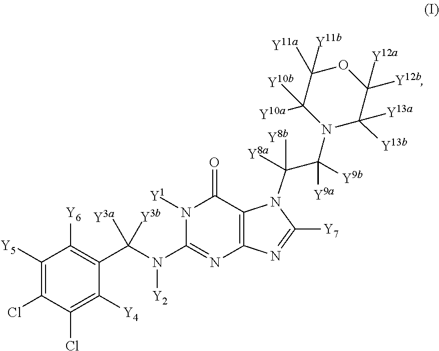

or a pharmaceutically acceptable salt thereof, wherein:

each of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} independently selected from hydrogen or deuterium; and

at least one of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} deuterium.

The invention excludes the compound with Y¹, Y², Y^{3a}, Y^{3b}, Y⁴, Y⁵, Y⁶, Y⁷, Y^{8a}, Y^{8b}, Y^{9a}, Y^{9b} as hydrogen and Y^{10a}, Y^{10b}, Y^{11a}, Y^{11b}, Y^{12a}, Y^{12b}, Y^{13a} and Y^{13b} as deuterium.

The invention further relates to a pharmaceutical composition including a compound of the invention and a pharmaceutically acceptable excipient.

The invention also relates to a method of treating or reducing the likelihood of developing a C. diff-associated disease by administering to an animal in need thereof a therapeutically effective amount of a compound of the invention. Exemplary C. diff-associated diseases include C. diff.-associated diarrhea or C. diff-associated colitis. In certain embodiments, the animal is at increased risk for C. diff infection, as described herein.

The invention further relates to a method of inhibiting growth of C. diff in vitro, the method comprising contacting, in an appropriate culture medium in the absence of oxygen, C. diff. with an effective amount of a compound of the invention.

Compounds of the invention may be used to treat or reduce the likelihood of developing a C. diff.-associated disease.

### DETAILED DESCRIPTION OF THE INVENTION

It will be understood that particular embodiments described herein are shown by way of illustration and not as limitation of the invention.

### Definitions

In the context of inhibiting bacterial growth, an “effective amount” of a compound is sufficient to reduce cellular growth rate. In some embodiments, an effective amount reduces cellular growth rate by at least 50%, by at least 60%, by at least 70%, or by at least 80%.

The term “inhibiting” means that cellular growth rate is reduced. In some embodiments, cellular growth rate is reduced by at least 50%, by at least 60%, by at least 70%, or by at least 80%. In certain embodiments, cellular growth can be inhibited by 90%, 95%, or 99% or more.

By “a subject susceptible to a C. diff. infection” is meant an animal (e.g., a mammal), including, but not limited to, humans, non-human primates, horses, cows, sheep, goats, dogs, cats, rats, mice, rabbits, and the like that is at increased risk, relative to the general population, of contracting a C. diff. infection. Examples of such subjects include those that have recently undergone antibiotic treatment for another bacterial infection, the young and the elderly. Such subjects can be identified using methods known to one of ordinary skill in the art.

By “pharmaceutically acceptable salts” are meant those derived from pharmaceutically acceptable inorganic and organic bases. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like. Additional salts include nontoxic ammonium, quaternary ammonium, and amine cations, including, but not limited to, ammonium, tetramethylammonium, tetraethylammonium, methylamine, dimethylamine, trimethylamine, triethylamine, ethylamine and the like. Pharmaceutically acceptable cations are those salt-forming ions with a positive charge. References hereinafter to a compound according to the invention include compounds of the general formulae shown, as well as their pharmaceutically acceptable salts.

The term “pharmaceutical composition,” as used herein, represents a composition containing a compound described herein, formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers to any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

By “therapeutically effective amount” is meant an amount which, when administered to an animal in need, will alleviate at least some of the symptoms of C. diff. infection. In the context of prophylaxis, a “therapeutically effective amount” is an amount which, when administered to a subject susceptible to C. diff infection, will help inhibit or reduce the likelihood of such an infection.

The term “reducing the likelihood of developing,” as used herein, refers to prophylactic treatment or treatment resulting in a reduction (e.g., at least 5%, 10%, 15%, 20%, 25%, 30%, 35%, 40%, 50%, 60%, 70%, 80%, 90%, or 95%) for a subject or a patient population in the chance or rate of developing a C. diff-associated disease by administering a compound of the invention compared to a subject or patient population not receiving the compound. Preventive treatment can be initiated, for example, prior to (“pre-exposure prophylaxis”) or following (“post-exposure prophylaxis”) an event that precedes the onset of C. diff. infection. Preventive treatment that includes administration of a compound of the invention, or a pharmaceutical composition thereof, can be acute, short-term, or chronic. The doses administered may be varied during the course of preventive treatment. The term also includes prevention of activity in vitro.

As used herein, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Any atom in the compounds of this invention not identified as a particular isotope implies the representation of any stable isotope of that atom. Unless specified otherwise, a position nominated as “H” or “hydrogen” is regarded to have hydrogen at its natural abundance isotopic composition.

At a given position in a molecule, the term “deuterium enrichment” indicates the percentage of incorporation of deuterium replacing hydrogen. As an example, at a given position, 1% of deuterium enrichment signifies 1% of molecules in a given sample containing deuterium at the specified position. The deuterium enrichment can be calculated by conventional analytical methods known to one of ordinary skill in the art, including mass spectrometry and nuclear magnetic resonance spectroscopy.

When used to describe a given position in a molecule or represent a given position in a drawing of a molecular structure, the term “is/are deuterium” is employed to mean that the specified position is enriched with deuterium at a level above the naturally occurring distribution of deuterium. In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

At a particular position normally filled by hydrogen, the term “isotopic enrichment factor” is defined to be the ratio between the abundance of deuterium and the natural abundance of deuterium at that position. For instance, an isotopic enrichment factor being 3500 indicates the amount of deuterium at the particular position 3500 times the natural abundance of deuterium, meaning 52.5% of the compounds have deuterium at the particular position (i.e., 52.5% deuterium incorporation at the specified position). In the oceans of Earth, the abundance of deuterium is roughly one atom in 6500 hydrogen atoms (about 154 parts per million (ppm)). Deuterium thus amounts to approximately 0.015% (or 0.030% on a weight basis) of all naturally occurring hydrogen atoms in the oceans of Earth; the abundance varies slightly from one kind of natural water to another.

The details of one or more embodiments of the invention are set forth in the accompanying description below. Other features, objects, and advantages of the invention will be apparent from the description and from the claims.

### Therapeutic Compounds

The compounds or pharmaceutically acceptable salts and the pharmaceutical compositions as described herein have activity as C. diff. inhibitors.

When referring to any compound of the invention, including Compound I-1, a compound represented by Formula I, or a pharmaceutically acceptable salt thereof, the term “compound” is used to refer to a collection of molecules having an identical chemical structure, barring a possible isotopic variation among the constituent hydrogen atoms of the molecules. A number of factors will influence the relative amount of isotopic variation in a compound of this invention, such as the isotopic purity of deuterated reagents applied to make the compound and the efficiency of deuterium incorporation in the various synthesis steps for preparing the compound.

Herein, both “D” and “d” are used to refer to deuterium and “H” to hydrogen.

Herein, “substituted with deuterium” is used to stand for one or more hydrogen atoms replaced with a corresponding number of deuterium atoms.

In one aspect, the present invention relates to a compound represented by Formula I:

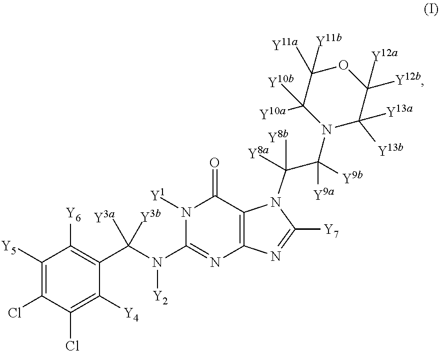

or a pharmaceutically acceptable salt thereof, wherein:

each of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} independently selected from hydrogen or deuterium; and

at least one of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} is deuterium.

The invention excludes the compound with Y¹, Y², Y^{3a}, Y^{3b}, Y⁴, Y⁵, Y⁶, Y⁷, Y^{8a}, Y^{8b}, Y^{9a}, Y^{9b} as hydrogen and Y^{10a}, Y^{10b}, Y^{11a}, Y^{11b}, Y^{12a}, Y^{12b}, Y^{13a} and Y^{13b} as deuterium.

In some embodiments of Formula I, at least one of Y⁴, Y⁵, Y⁶, and Y⁷ is deuterium. In particular, the compound of Formula I is selected from any one of the compounds set forth in the table below:

| Compound | Y⁴ | Y⁵ | Y⁶ | Y⁷ |
|---|---|---|---|---|
| 1 | D | H | H | H |
| 2 | H | D | H | H |
| 3 | H | H | D | H |
| 4 | H | H | H | D |
| 5 | D | D | H | H |
| 6 | D | H | D | H |
| 7 | D | H | H | D |
| 8 | H | D | D | H |
| 9 | H | D | H | D |
| 10 | H | H | D | D |
| 11 | D | D | D | H |
| 12 | D | D | H | D |
| 13 | D | H | D | D |
| 14 | H | D | D | D |
| 15 | D | D | D | D |

wherein Y¹, Y², Y^{3a}, Y^{3b}, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} are all H.

In some embodiments of Formula I, at least one of Y^{3a}, Y^{3b}, Y^{8a}, Y^{8b}, Y^{9a} and Y^{9b} is deuterium; In particular, the compound of Formula I is selected from any one of the compounds set forth in the table below:

| Compound | Y^{3a} | Y^{3b} | Y^{8a} | Y^{8b} | Y^{9a} | Y^{9b} |
|---|---|---|---|---|---|---|
| 16 | D | H | H | H | H | H |
| 17 | H | H | D | H | H | H |
| 18 | H | H | H | D | H | H |
| 19 | H | H | H | H | D | H |
| 20 | H | H | H | H | H | D |
| 21 | D | D | H | H | H | H |
| 22 | D | H | D | H | H | H |
| 23 | D | H | H | D | H | H |
| 24 | D | H | H | H | D | H |
| 25 | D | H | H | H | H | D |
| 26 | H | H | D | D | H | H |
| 27 | H | H | D | H | D | H |
| 28 | H | H | D | H | H | D |
| 29 | H | H | H | D | D | H |
| 30 | H | H | H | D | H | D |
| 31 | H | H | H | H | D | D |
| 32 | D | D | D | H | H | H |
| 33 | D | D | H | D | H | H |
| 34 | D | D | H | H | D | H |
| 35 | D | D | H | H | H | D |
| 36 | D | H | D | D | H | H |
| 37 | D | H | D | H | D | H |
| 38 | D | H | D | H | H | D |
| 39 | D | H | H | D | D | H |
| 40 | D | H | H | D | H | D |
| 41 | D | H | H | H | D | D |
| 42 | H | H | D | D | D | H |
| 43 | H | H | D | D | H | D |
| 44 | H | H | D | H | D | D |
| 45 | H | H | H | D | D | D |
| 46 | D | D | D | D | H | H |
| 47 | D | D | D | H | D | H |
| 48 | D | D | D | H | H | D |
| 49 | D | D | H | D | D | H |
| 50 | D | D | H | D | H | D |
| 51 | D | D | H | H | D | D |
| 52 | D | H | D | D | D | H |
| 53 | D | H | D | D | H | D |
| 54 | D | H | D | H | D | D |
| 55 | D | H | H | D | D | D |
| 56 | H | H | D | D | D | D |
| 57 | D | D | D | D | D | H |
| 58 | D | D | D | D | H | D |
| 59 | D | D | D | H | D | D |
| 60 | D | D | H | D | D | D |
| 61 | D | H | D | D | D | D |
| 62 | D | D | D | D | D | D |

wherein Y¹, Y², Y⁴, Y⁵, Y⁶, Y⁷, Y^{10a}, Y^{10b}, Y^{11a}, Y^{11b}, Y^{12a}, Y^{12b}, Y^{13a} and Y^{13b} are all H.

In other embodiments of the invention, the compound of Formula I is selected from any one of the compounds set forth in the table below:

| | Y^{10a}, | Y^{11a}, | Y^{12a}, | Y^{13a}, | Y^{3a}, | Y^{8a}, | Y^{9a} | | |
|---|---|---|---|---|---|---|---|---|---|
| Compound | Y^{10b} | Y^{11b} | Y^{12b} | Y^{13b} | Y^{3b} | Y^{8b} | Y^{9b}, | Y⁵ | Y⁷ |
| 63 | D | H | H | D | H | H | H | H | H |
| 64 | D | H | H | H | D | H | H | H | H |
| 65 | D | H | H | H | H | D | H | H | H |
| 66 | D | H | H | H | H | H | D | H | H |
| 67 | D | H | H | H | H | H | H | D | H |
| 68 | D | H | H | H | H | H | H | H | D |
| 69 | H | H | D | D | H | H | H | H | H |
| 70 | H | H | D | H | D | H | H | H | H |
| 71 | H | H | D | H | H | D | H | H | H |
| 72 | H | H | D | H | H | H | D | H | H |
| 73 | H | H | D | H | H | H | H | D | H |
| 74 | H | H | D | H | H | H | H | H | D |
| 75 | H | H | H | D | H | H | D | H | H |
| 76 | H | H | H | D | H | H | H | D | H |
| 77 | H | H | H | D | H | H | H | H | D |
| 78 | H | H | H | H | D | H | H | D | H |
| 79 | H | H | H | H | H | D | H | D | H |
| 80 | H | H | H | H | H | D | H | H | D |
| 81 | H | H | H | H | H | H | D | H | D |
| 82 | D | D | H | D | H | H | H | H | H |
| 83 | D | D | H | H | D | H | H | H | H |
| 84 | D | D | H | H | H | D | H | H | H |
| 85 | D | D | H | H | H | H | D | H | H |
| 86 | D | D | H | H | H | H | H | D | H |
| 87 | D | D | H | H | H | H | H | H | D |
| 88 | D | H | D | D | H | H | H | H | H |
| 89 | D | H | D | H | D | H | H | H | H |
| 90 | D | H | D | H | H | D | H | H | H |
| 91 | D | H | D | H | H | H | D | H | H |
| 92 | D | H | D | H | H | H | H | D | H |
| 93 | D | H | D | H | H | H | H | H | D |
| 94 | D | H | H | D | D | H | H | H | H |
| 95 | D | H | H | D | H | D | H | H | H |
| 96 | D | H | H | D | H | H | D | H | H |
| 97 | D | H | H | D | H | H | H | D | H |
| 98 | D | H | H | D | H | H | H | H | D |
| 99 | D | H | H | H | D | D | H | H | H |
| 100 | D | H | H | H | D | H | D | H | H |
| 101 | D | H | H | H | D | H | H | D | H |
| 102 | D | H | H | H | D | H | H | H | D |
| 103 | D | H | H | H | H | D | D | H | H |
| 104 | D | H | H | H | H | D | H | D | H |
| 105 | D | H | H | H | H | D | H | H | D |
| 106 | D | H | H | H | H | H | D | D | H |
| 107 | D | H | H | H | H | H | D | H | D |
| 108 | D | H | H | H | H | H | H | D | D |
| 109 | H | H | D | D | D | H | H | H | H |
| 110 | H | H | D | D | H | D | H | H | H |
| 111 | H | H | D | D | H | H | D | H | H |
| 112 | H | H | D | D | H | H | H | D | H |
| 113 | H | H | D | D | H | H | H | H | D |
| 114 | H | H | D | H | D | D | H | H | H |
| 115 | H | H | D | H | D | H | D | H | H |
| 116 | H | H | D | H | D | H | H | D | H |
| 117 | H | H | D | H | D | H | H | H | D |
| 118 | H | H | D | H | H | D | D | H | H |
| 119 | H | H | D | H | H | D | H | D | H |
| 120 | H | H | D | H | H | D | H | H | D |
| 121 | H | H | D | H | H | H | D | D | H |
| 122 | H | H | D | H | H | H | D | H | D |
| 123 | H | H | D | H | H | H | H | D | D |
| 124 | H | H | H | D | D | H | D | H | H |
| 125 | H | H | H | D | D | H | H | D | H |
| 126 | H | H | H | D | D | H | H | H | D |
| 127 | H | H | H | D | H | D | D | H | H |
| 128 | H | H | H | D | H | D | H | D | H |
| 129 | H | H | H | D | H | D | H | H | D |
| 130 | H | H | H | D | H | H | D | D | H |
| 131 | H | H | H | D | H | H | D | H | D |
| 132 | H | H | H | D | H | H | H | D | D |
| 133 | H | H | H | H | D | D | H | D | H |
| 134 | H | H | H | H | D | D | H | H | D |
| 135 | H | H | H | H | D | H | D | D | H |
| 136 | H | H | H | H | D | H | D | H | D |
| 137 | H | H | H | H | D | H | H | D | D |
| 138 | H | H | H | H | H | D | D | D | H |
| 139 | H | H | H | H | H | D | D | H | D |
| 140 | H | H | H | H | H | D | H | D | D |
| 141 | H | H | H | H | H | H | D | D | D |
| 142 | D | D | D | H | D | H | H | H | H |
| 143 | D | D | D | H | H | D | H | H | H |
| 144 | D | D | D | H | H | H | D | H | H |
| 145 | D | D | D | H | H | H | H | D | H |
| 146 | D | D | D | H | H | H | H | H | D |
| 147 | D | D | H | D | D | H | H | H | H |
| 148 | D | D | H | D | H | D | H | H | H |
| 149 | D | D | H | D | H | H | D | H | H |
| 150 | D | D | H | D | H | H | H | D | H |
| 151 | D | D | H | D | H | H | H | H | D |
| 152 | D | D | H | H | D | D | H | H | H |
| 153 | D | D | H | H | D | H | D | H | H |
| 154 | D | D | H | H | D | H | H | D | H |
| 155 | D | D | H | H | D | H | H | H | D |
| 156 | D | D | H | H | H | D | D | H | H |
| 157 | D | D | H | H | H | D | H | D | H |
| 158 | D | D | H | H | H | D | H | H | D |
| 159 | D | D | H | H | H | H | D | D | H |
| 160 | D | D | H | H | H | H | D | H | D |
| 161 | D | D | H | H | H | H | H | D | D |
| 162 | D | H | D | D | D | H | H | H | H |
| 163 | D | H | D | D | H | D | H | H | H |
| 164 | D | H | D | D | H | H | D | H | H |
| 165 | D | H | D | D | H | H | H | D | H |
| 166 | D | H | D | D | H | H | H | H | D |
| 167 | D | H | D | H | D | D | H | H | H |
| 168 | D | H | D | H | D | H | D | H | H |
| 169 | D | H | D | H | D | H | H | D | H |
| 170 | D | H | D | H | D | H | H | H | D |
| 171 | D | H | D | H | H | D | D | H | H |
| 172 | D | H | D | H | H | D | H | D | H |
| 173 | D | H | D | H | H | D | H | H | D |
| 174 | D | H | D | H | H | H | D | D | H |
| 175 | D | H | D | H | H | H | D | H | D |
| 176 | D | H | D | H | H | H | H | D | D |
| 177 | D | H | H | D | D | D | H | H | H |
| 178 | D | H | H | D | D | H | D | H | H |
| 179 | D | H | H | D | D | H | H | D | H |
| 180 | D | H | H | D | D | H | H | H | D |
| 181 | D | H | H | D | H | D | D | H | H |
| 182 | D | H | H | D | H | D | H | D | H |
| 183 | D | H | H | D | H | D | H | H | D |
| 184 | D | H | H | D | H | H | D | D | H |
| 185 | D | H | H | D | H | H | D | H | D |
| 186 | D | H | H | D | H | H | H | D | D |
| 187 | D | H | H | H | D | D | D | H | H |
| 188 | D | H | H | H | D | D | H | D | H |
| 189 | D | H | H | H | D | D | H | H | D |
| 190 | D | H | H | H | D | H | D | D | H |
| 191 | D | H | H | H | D | H | D | H | D |
| 192 | D | H | H | H | D | H | H | D | D |
| 193 | D | H | H | H | H | D | D | D | H |
| 194 | D | H | H | H | H | D | D | H | D |
| 195 | D | H | H | H | H | D | H | D | D |
| 196 | D | H | H | H | H | H | D | D | D |
| 197 | H | H | D | D | D | D | H | H | H |
| 198 | H | H | D | D | D | H | D | H | H |
| 199 | H | H | D | D | D | H | H | D | H |
| 200 | H | H | D | D | D | H | H | H | D |
| 201 | H | H | D | D | H | D | D | H | H |
| 202 | H | H | D | D | H | D | H | D | H |
| 203 | H | H | D | D | H | D | H | H | D |
| 204 | H | H | D | D | H | H | D | D | H |
| 205 | H | H | D | D | H | H | D | H | D |
| 206 | H | H | D | D | H | H | H | D | D |
| 207 | H | H | D | H | D | D | D | H | H |
| 208 | H | H | D | H | D | D | H | D | H |
| 209 | H | H | D | H | D | D | H | H | D |
| 210 | H | H | D | H | D | H | D | D | H |
| 211 | H | H | D | H | D | H | D | H | D |
| 212 | H | H | D | H | D | H | H | D | D |
| 213 | H | H | D | H | H | D | D | D | H |
| 214 | H | H | D | H | H | D | D | H | D |
| 215 | H | H | D | H | H | D | H | D | D |
| 216 | H | H | D | H | H | H | D | D | D |
| 217 | H | H | H | D | D | D | D | H | H |
| 218 | H | H | H | D | D | D | H | D | H |
| 219 | H | H | H | D | D | D | H | H | D |
| 220 | H | H | H | D | D | H | D | D | H |
| 221 | H | H | H | D | D | H | D | H | D |
| 222 | H | H | H | D | D | H | H | D | D |
| 223 | H | H | H | D | H | D | D | D | H |
| 224 | H | H | H | D | H | D | D | H | D |
| 225 | H | H | H | D | H | D | H | D | D |
| 226 | H | H | H | D | H | H | D | D | D |
| 227 | H | H | H | H | D | D | D | D | H |
| 228 | H | H | H | H | D | D | D | H | D |
| 229 | H | H | H | H | D | D | H | D | D |
| 230 | H | H | H | H | D | H | D | D | D |
| 231 | H | H | H | H | H | D | D | D | D |
| 232 | D | D | D | D | D | H | H | H | H |
| 233 | D | D | D | D | H | D | H | H | H |
| 234 | D | D | D | D | H | H | D | H | H |
| 235 | D | D | D | D | H | H | H | D | H |
| 236 | D | D | D | D | H | H | H | H | D |
| 237 | D | D | D | H | D | D | H | H | H |
| 238 | D | D | D | H | D | H | D | H | H |
| 239 | D | D | D | H | D | H | H | D | H |
| 240 | D | D | D | H | D | H | H | H | D |
| 241 | D | D | D | H | H | D | D | H | H |
| 242 | D | D | D | H | H | D | H | D | H |
| 243 | D | D | D | H | H | D | H | H | D |
| 244 | D | D | D | H | H | H | D | D | H |
| 245 | D | D | D | H | H | H | D | H | D |
| 246 | D | D | D | H | H | H | H | D | D |
| 247 | D | D | H | D | D | D | H | H | H |
| 248 | D | D | H | D | D | H | D | H | H |
| 249 | D | D | H | D | D | H | H | D | H |
| 250 | D | D | H | D | D | H | H | H | D |
| 251 | D | D | H | D | H | D | D | H | H |
| 252 | D | D | H | D | H | D | H | D | H |
| 253 | D | D | H | D | H | D | H | H | D |
| 254 | D | D | H | D | H | H | D | D | H |
| 255 | D | D | H | D | H | H | D | H | D |
| 256 | D | D | H | D | H | H | H | D | D |
| 257 | D | D | H | H | D | D | D | H | H |
| 258 | D | D | H | H | D | D | H | D | H |
| 259 | D | D | H | H | D | D | H | H | D |
| 260 | D | D | H | H | D | H | D | D | H |
| 261 | D | D | H | H | D | H | D | H | D |
| 262 | D | D | H | H | D | H | H | D | D |
| 263 | D | D | H | H | H | D | D | D | H |
| 264 | D | D | H | H | H | D | D | H | D |
| 265 | D | D | H | H | H | D | H | D | D |
| 266 | D | D | H | H | H | H | D | D | D |
| 267 | D | H | D | D | D | D | H | H | H |
| 268 | D | H | D | D | D | H | D | H | H |
| 269 | D | H | D | D | D | H | H | D | H |
| 270 | D | H | D | D | D | H | H | H | D |
| 271 | D | H | D | D | H | D | D | H | H |
| 272 | D | H | D | D | H | D | H | D | H |
| 273 | D | H | D | D | H | D | H | H | D |
| 274 | D | H | D | D | H | H | D | D | H |
| 275 | D | H | D | D | H | H | D | H | D |
| 276 | D | H | D | D | H | H | H | D | D |
| 277 | D | H | D | H | D | D | D | H | H |
| 278 | D | H | D | H | D | D | H | D | H |
| 279 | D | H | D | H | D | D | H | H | D |
| 280 | D | H | D | H | D | H | D | D | H |
| 281 | D | H | D | H | D | H | D | H | D |
| 282 | D | H | D | H | D | H | H | D | D |
| 283 | D | H | D | H | H | D | D | D | H |
| 284 | D | H | D | H | H | D | D | H | D |
| 285 | D | H | D | H | H | D | H | D | D |
| 286 | D | H | D | H | H | H | D | D | D |
| 287 | D | H | H | D | D | D | D | H | H |
| 288 | D | H | H | D | D | D | H | D | H |
| 289 | D | H | H | D | D | D | H | H | D |
| 290 | D | H | H | D | D | H | D | D | H |
| 291 | D | H | H | D | D | H | D | H | D |
| 292 | D | H | H | D | D | H | H | D | D |
| 293 | D | H | H | D | H | D | D | D | H |
| 294 | D | H | H | D | H | D | D | H | D |
| 295 | D | H | H | D | H | D | H | D | D |
| 296 | D | H | H | D | H | H | D | D | D |
| 297 | D | H | H | H | D | D | D | D | H |
| 298 | D | H | H | H | D | D | D | H | D |
| 299 | D | H | H | H | D | D | H | D | D |
| 300 | D | H | H | H | D | H | D | D | D |
| 301 | D | H | H | H | H | D | D | D | D |
| 302 | H | H | D | D | D | D | D | H | H |
| 303 | H | H | D | D | D | D | H | D | H |
| 304 | H | H | D | D | D | D | H | H | D |
| 305 | H | H | D | D | D | H | D | D | H |
| 306 | H | H | D | D | D | H | D | H | D |
| 307 | H | H | D | D | D | H | H | D | D |
| 308 | H | H | D | D | H | D | D | D | H |
| 309 | H | H | D | D | H | D | D | H | D |
| 310 | H | H | D | D | H | D | H | D | D |
| 311 | H | H | D | D | H | H | D | D | D |
| 312 | H | H | D | H | D | D | D | D | H |
| 313 | H | H | D | H | D | D | D | H | D |
| 314 | H | H | D | H | D | D | H | D | D |
| 315 | H | H | D | H | D | H | D | D | D |
| 316 | H | H | D | H | H | D | D | D | D |
| 317 | H | H | H | D | D | D | D | D | H |
| 318 | H | H | H | D | D | D | D | H | D |
| 319 | H | H | H | D | D | D | H | D | D |
| 320 | H | H | H | D | D | H | D | D | D |
| 321 | H | H | H | D | H | D | D | D | D |
| 322 | H | H | H | H | D | D | D | D | D |
| 323 | D | D | D | D | D | D | H | H | H |
| 324 | D | D | D | D | D | H | D | H | H |
| 325 | D | D | D | D | D | H | H | D | H |
| 326 | D | D | D | D | D | H | H | H | D |
| 327 | D | D | D | D | H | D | D | H | H |
| 328 | D | D | D | D | H | D | H | D | H |
| 329 | D | D | D | D | H | D | H | H | D |
| 330 | D | D | D | D | H | H | D | D | H |
| 331 | D | D | D | D | H | H | D | H | D |
| 332 | D | D | D | D | H | H | H | D | D |
| 333 | D | D | D | H | D | D | D | H | H |
| 334 | D | D | D | H | D | D | H | D | H |
| 335 | D | D | D | H | D | D | H | H | D |
| 336 | D | D | D | H | D | H | D | D | H |
| 337 | D | D | D | H | D | H | D | H | D |
| 338 | D | D | D | H | D | H | H | D | D |
| 339 | D | D | D | H | H | D | D | D | H |
| 340 | D | D | D | H | H | D | D | H | D |
| 341 | D | D | D | H | H | D | H | D | D |
| 342 | D | D | D | H | H | H | D | D | D |
| 343 | D | D | H | D | D | D | D | H | H |
| 344 | D | D | H | D | D | D | H | D | H |
| 345 | D | D | H | D | D | D | H | H | D |
| 346 | D | D | H | D | D | H | D | D | H |
| 347 | D | D | H | D | D | H | D | H | D |
| 348 | D | D | H | D | D | H | H | D | D |
| 349 | D | D | H | D | H | D | D | D | H |
| 350 | D | D | H | D | H | D | D | H | D |
| 351 | D | D | H | D | H | D | H | D | D |
| 352 | D | D | H | D | H | H | D | D | D |
| 353 | D | D | H | H | D | D | D | D | H |
| 354 | D | D | H | H | D | D | D | H | D |
| 355 | D | D | H | H | D | D | H | D | D |
| 356 | D | D | H | H | D | H | D | D | D |
| 357 | D | D | H | H | H | D | D | D | D |
| 358 | D | H | D | D | D | D | D | H | H |
| 359 | D | H | D | D | D | D | H | D | H |
| 360 | D | H | D | D | D | D | H | H | D |
| 361 | D | H | D | D | D | H | D | D | H |
| 362 | D | H | D | D | D | H | D | H | D |
| 363 | D | H | D | D | D | H | H | D | D |
| 364 | D | H | D | D | H | D | D | D | H |
| 365 | D | H | D | D | H | D | D | H | D |
| 366 | D | H | D | D | H | D | H | D | D |
| 367 | D | H | D | D | H | H | D | D | D |
| 368 | D | H | D | H | D | D | D | D | H |
| 369 | D | H | D | H | D | D | D | H | D |
| 370 | D | H | D | H | D | D | H | D | D |
| 371 | D | H | D | H | D | H | D | D | D |
| 372 | D | H | D | H | H | D | D | D | D |
| 373 | D | H | H | D | D | D | D | D | H |
| 374 | D | H | H | D | D | D | D | H | D |
| 375 | D | H | H | D | D | D | H | D | D |
| 376 | D | H | H | D | D | H | D | D | D |
| 377 | D | H | H | D | H | D | D | D | D |
| 378 | D | H | H | H | D | D | D | D | D |
| 379 | H | H | D | D | D | D | D | D | H |
| 380 | H | H | D | D | D | D | D | H | D |
| 381 | H | H | D | D | D | D | H | D | D |
| 382 | H | H | D | D | D | H | D | D | D |
| 383 | H | H | D | D | H | D | D | D | D |
| 384 | H | H | D | H | D | D | D | D | D |
| 385 | H | H | H | D | D | D | D | D | D |
| 386 | D | D | D | D | D | D | D | H | H |
| 387 | D | D | D | D | D | D | H | D | H |
| 388 | D | D | D | D | D | D | H | H | D |
| 389 | D | D | D | D | D | H | D | D | H |
| 390 | D | D | D | D | D | H | D | H | D |
| 391 | D | D | D | D | D | H | H | D | D |
| 392 | D | D | D | D | H | D | D | D | H |
| 393 | D | D | D | D | H | D | D | H | D |
| 394 | D | D | D | D | H | D | H | D | D |
| 395 | D | D | D | D | H | H | D | D | D |
| 396 | D | D | D | H | D | D | D | D | H |
| 397 | D | D | D | H | D | D | D | H | D |
| 398 | D | D | D | H | D | D | H | D | D |
| 399 | D | D | D | H | D | H | D | D | D |
| 400 | D | D | D | H | H | D | D | D | D |
| 401 | D | D | H | D | D | D | D | D | H |
| 402 | D | D | H | D | D | D | D | H | D |
| 403 | D | D | H | D | D | D | H | D | D |
| 404 | D | D | H | D | D | H | D | D | D |
| 405 | D | D | H | D | H | D | D | D | D |
| 406 | D | D | H | H | D | D | D | D | D |
| 407 | D | H | D | D | D | D | D | D | H |
| 408 | D | H | D | D | D | D | D | H | D |
| 409 | D | H | D | D | D | D | H | D | D |
| 410 | D | H | D | D | D | H | D | D | D |
| 411 | D | H | D | D | H | D | D | D | D |
| 412 | D | H | D | H | D | D | D | D | D |
| 413 | D | H | H | D | D | D | D | D | D |
| 414 | H | H | D | D | D | D | D | D | D |
| 415 | D | D | D | D | D | D | D | D | H |
| 416 | D | D | D | D | D | D | D | H | D |
| 417 | D | D | D | D | D | D | H | D | D |
| 418 | D | D | D | D | D | H | D | D | D |
| 419 | D | D | D | D | H | D | D | D | D |
| 420 | D | D | D | H | D | D | D | D | D |
| 421 | D | D | H | D | D | D | D | D | D |
| 422 | D | H | D | D | D | D | D | D | D |
| 423 | D | D | D | D | D | D | D | D | D |
| 424 | H | D | D | H | H | H | H | H | H |
| 425 | D | D | H | H | H | H | H | H | H |
| 426 | H | D | H | H | H | H | H | H | H |

wherein Y⁴ and Y⁶ are all H.

In particular embodiments of the invention, the compound of Formula I is selected from any one of the following compounds:

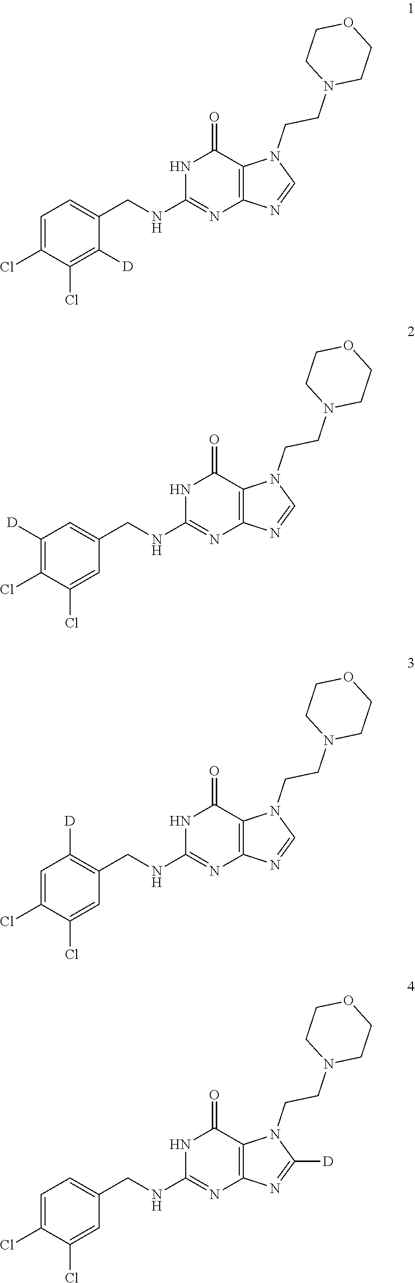 [SMILES: [2H]c1c(CNc2nc3ncn(CCN4CCOCC4)c3c(=O)[nH]2)ccc(Cl)c1Cl ; [2H]c1cc(CNc2nc3ncn(CCN4CCOCC4)c3c(=O)[nH]2)cc(Cl)c1Cl ; [2H]c1cc(Cl)c(Cl)cc1CNc1nc2ncn(CCN3CCOCC3)c2c(=O)[nH]1 ; [2H]c1nc2nc(NCc3ccc(Cl)c(Cl)c3)[nH]c(=O)c2n1CCN1CCOCC1]

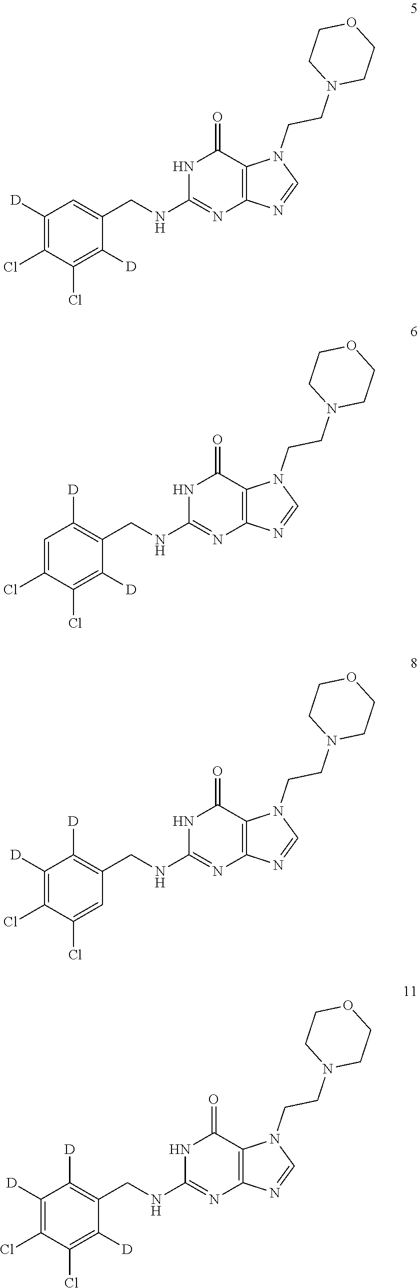 [SMILES: [2H]c1cc(CNc2nc3ncn(CCN4CCOCC4)c3c(=O)[nH]2)c([2H])c(Cl)c1Cl ; [2H]c1cc(Cl)c(Cl)c([2H])c1CNc1nc2ncn(CCN3CCOCC3)c2c(=O)[nH]1 ; [2H]c1c(CNc2nc3ncn(CCN4CCOCC4)c3c(=O)[nH]2)cc(Cl)c(Cl)c1[2H] ; [2H]c1c([2H])c(CNc2nc3ncn(CCN4CCOCC4)c3c(=O)[nH]2)c([2H])c(Cl)c1Cl]

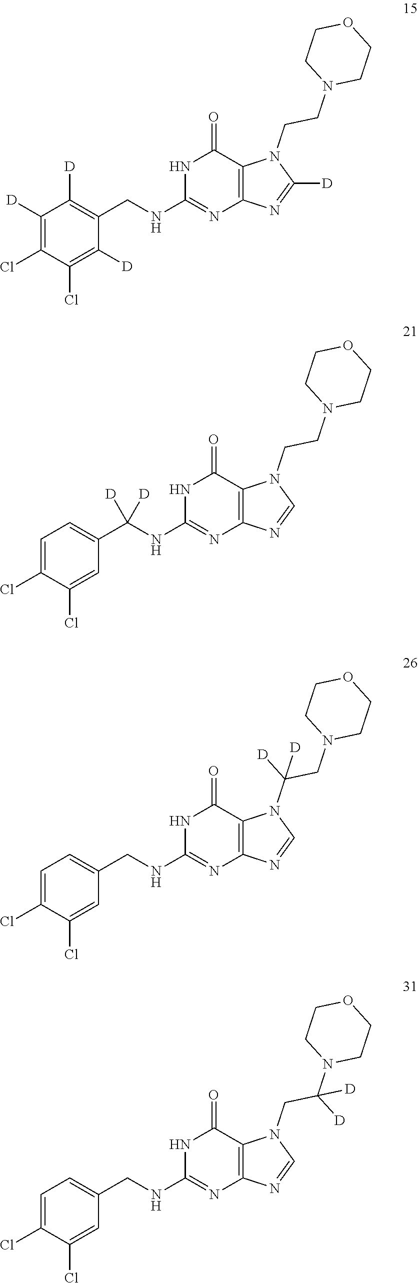 [SMILES: [2H]c1c([2H])c(CNc2nc3nc([2H])n(CCN4CCOCC4)c3c(=O)[nH]2)c([2H])c(Cl)c1Cl ; [2H]C([2H])(Nc1nc2ncn(CCN3CCOCC3)c2c(=O)[nH]1)c1ccc(Cl)c(Cl)c1 ; [2H]C([2H])(CN1CCOCC1)n1cnc2nc(NCc3ccc(Cl)c(Cl)c3)[nH]c(=O)c21 ; [2H]C([2H])(Cn1cnc2nc(NCc3ccc(Cl)c(Cl)c3)[nH]c(=O)c21)N1CCOCC1]

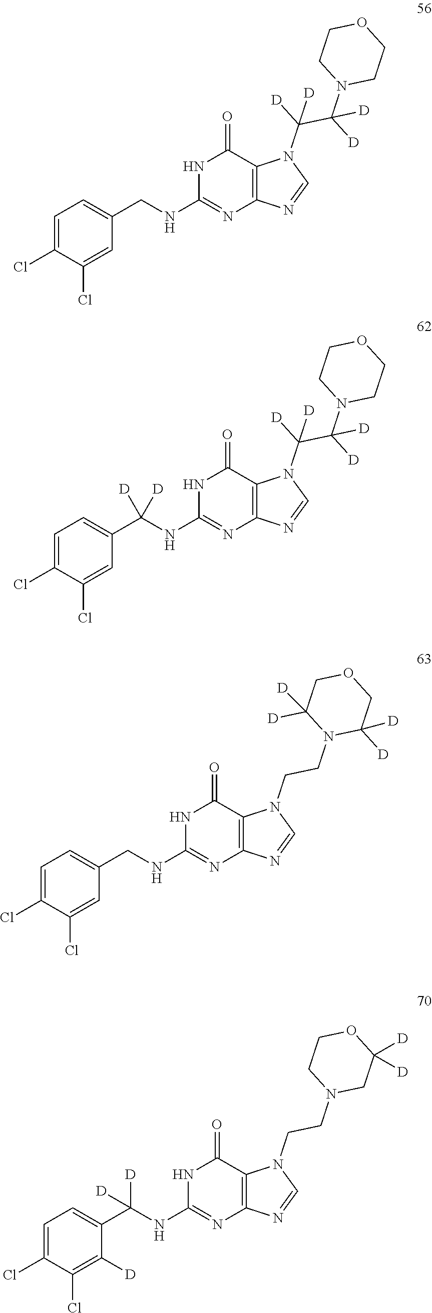 [SMILES: [2H]C([2H])(N1CCOCC1)C([2H])([2H])n1cnc2nc(NCc3ccc(Cl)c(Cl)c3)[nH]c(=O)c21 ; [2H]C([2H])(Nc1nc2ncn(C([2H])([2H])C([2H])([2H])N3CCOCC3)c2c(=O)[nH]1)c1ccc(Cl)c(Cl)c1 ; [2H]C1([2H])COCC([2H])([2H])N1CCn1cnc2nc(NCc3ccc(Cl)c(Cl)c3)[nH]c(=O)c21 ; [2H]c1c(C([2H])([2H])Nc2nc3ncn(CCN4CCOC([2H])([2H])C4)c3c(=O)[nH]2)ccc(Cl)c1Cl]

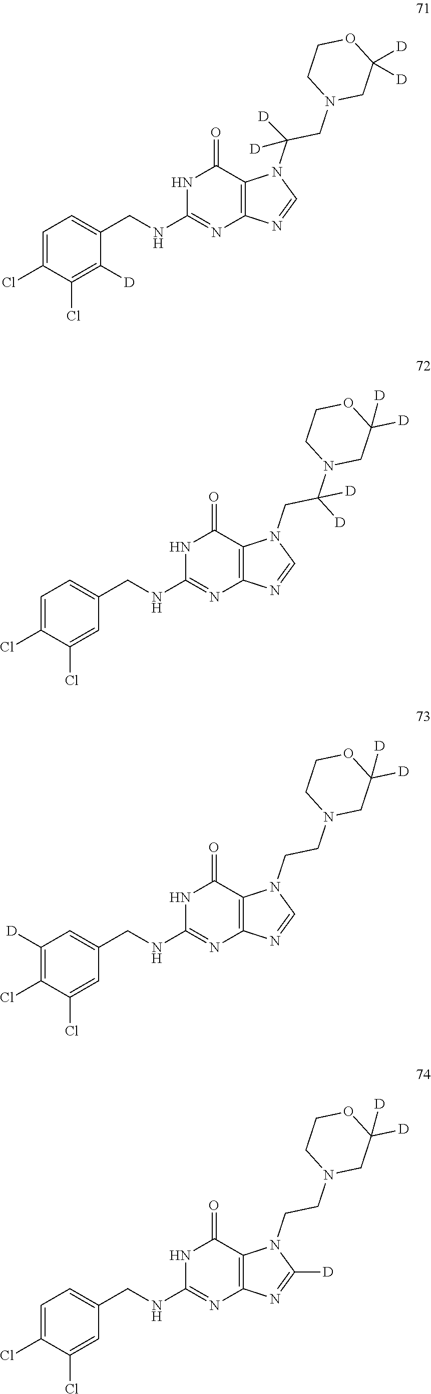 [SMILES: [2H]c1c(CNc2nc3ncn(C([2H])([2H])CN4CCOC([2H])([2H])C4)c3c(=O)[nH]2)ccc(Cl)c1Cl ; [2H]C1([2H])CN(C([2H])([2H])Cn2cnc3nc(NCc4ccc(Cl)c(Cl)c4)[nH]c(=O)c32)CCO1 ; [2H]c1cc(CNc2nc3ncn(CCN4CCOC([2H])([2H])C4)c3c(=O)[nH]2)cc(Cl)c1Cl ; [2H]c1nc2nc(NCc3ccc(Cl)c(Cl)c3)[nH]c(=O)c2n1CCN1CCOC([2H])([2H])C1]

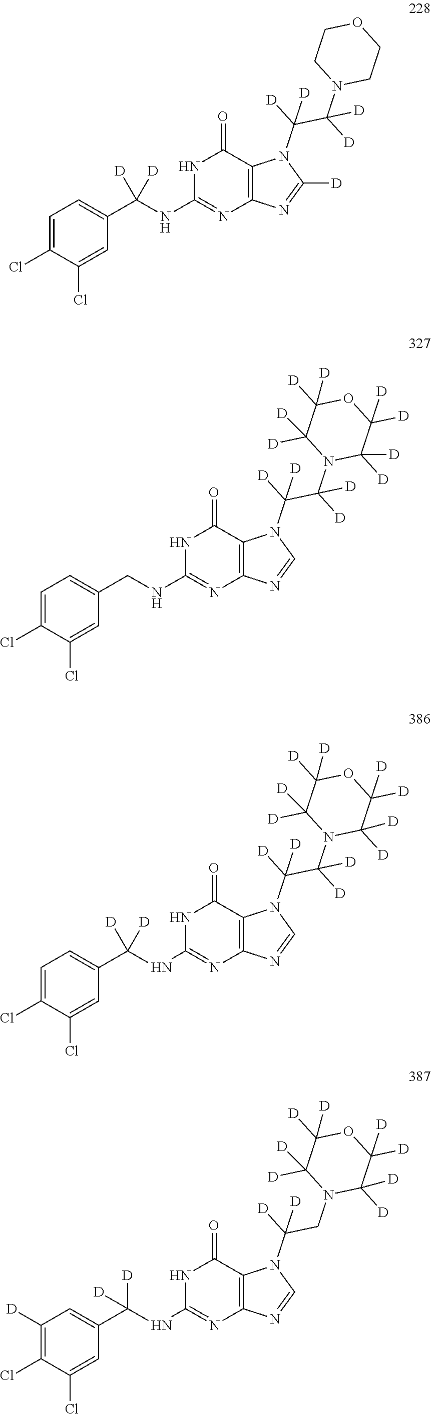 [SMILES: [2H]c1nc2nc(NC([2H])([2H])c3ccc(Cl)c(Cl)c3)[nH]c(=O)c2n1C([2H])([2H])C([2H])([2H])N1CCOCC1 ; [2H]C1([2H])OC([2H])([2H])C([2H])([2H])N(C([2H])([2H])C([2H])([2H])n2cnc3nc(NCc4ccc(Cl)c(Cl)c4)[nH]c(=O)c32)C1([2H])[2H] ; [2H]C([2H])(Nc1nc2ncn(C([2H])([2H])C([2H])([2H])N3C([2H])([2H])C([2H])([2H])OC([2H])([2H])C3([2H])[2H])c2c(=O)[nH]1)c1ccc(Cl)c(Cl)c1 ; [2H]c1cc(C([2H])([2H])Nc2nc3ncn(C([2H])([2H])CN4C([2H])([2H])C([2H])([2H])OC([2H])([2H])C4([2H])[2H])c3c(=O)[nH]2)cc(Cl)c1Cl]

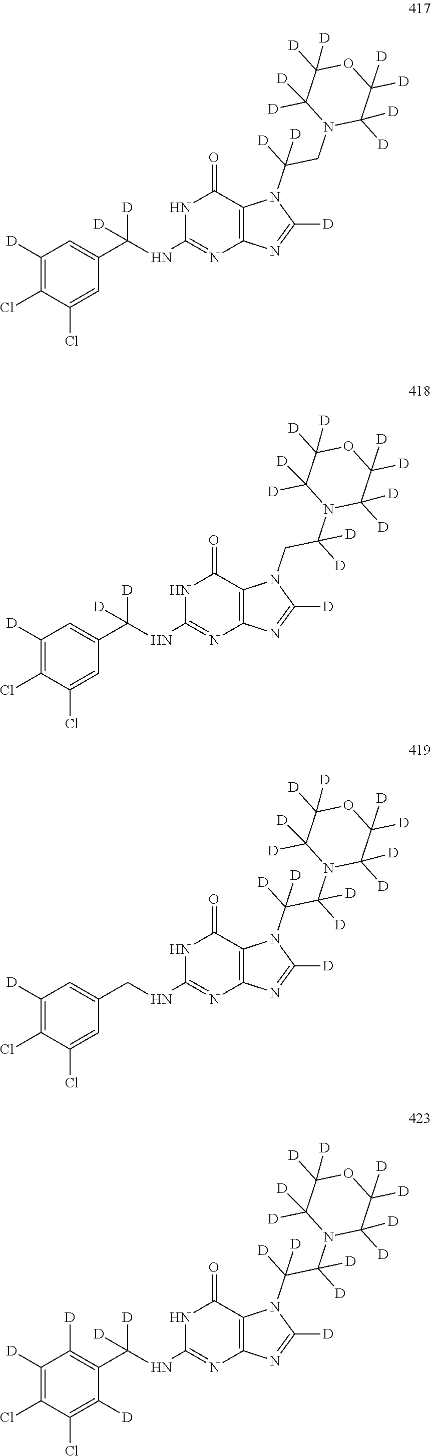

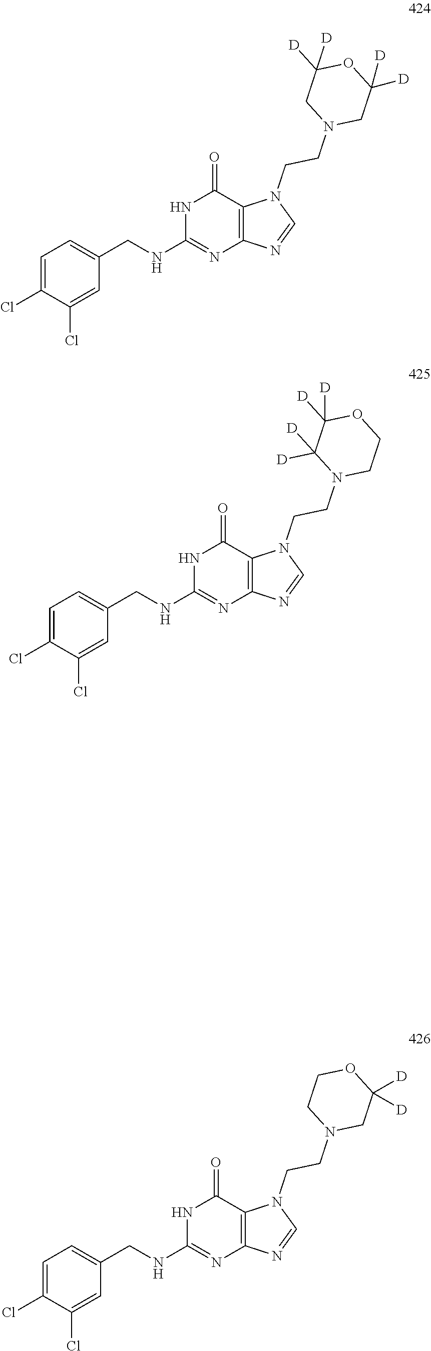 [SMILES: [2H]C1([2H])CN(CCn2cnc3nc(NCc4ccc(Cl)c(Cl)c4)[nH]c(=O)c32)CC([2H])([2H])O1 ; [2H]C1([2H])OCCN(CCn2cnc3nc(NCc4ccc(Cl)c(Cl)c4)[nH]c(=O)c32)C1([2H])[2H] ; [2H]C1([2H])CN(CCn2cnc3nc(NCc4ccc(Cl)c(Cl)c4)[nH]c(=O)c32)CCO1]

or a pharmaceutically acceptable salt thereof.

The low oral bioavailability of these compounds in mammals endows this class of agents with the characteristics advantageous for alleviating an intestinal infection, e.g., with C. diff. The bioavailability of the deuterated analogs may be the same or different from the bioavailability of the compounds that are not substituted with deuterium.

### Mechanism of Action

Without wishing to be bound by theory, the compounds target DNA polymerase III (pol III), an essential enzyme in DNA replication; thus, development of drug resistance will be minimized. The compounds can be used to circumvent the natural and acquired resistance of pathogenic C. diff. to conventional antimicrobials, for example vancomycin and metronidazole.

Genome sequence analysis has indicated that Gram-positive eubacteria of the so-called low G:C class, i.e., those with genomes containing a proportion of guanine+cytosine of less than 0.5, contain two types of DNA polymerase III (pol III): pol IIIC, encoded by a polC gene, and pol IIIE, encoded by one or more dnaE genes, (See, Wright, G. and Brown, N. Current Opinion in Anti-Infective Investigational Drugs 1:45-48 (1999) and Braithewaite, D. and Ito, J. Nucl. Acids Res. 21:787-802 (1993)). The compounds described herein were designed to inhibit either or both of the pol IIIC and pol IIIE enzymes.

Gram-positive pol IIIC and Gram-positive pol IIIE are enzymes that are both required for the replicative synthesis of DNA that accompanies the duplication of the Gram-positive bacterial chromosome. Gram-negative pol IIIE is the enzyme that is required for the replicative synthesis of DNA that accompanies the duplication of the Gram-negative bacterial chromosome. The compounds described herein mimic purine deoxyribonucleoside-5-triphosphates and physically inhibit the DNA polymerases. The mechanism of action of related N3-substituted pyrimidines is further described in U.S. Pat. No. 5,516,905. Because certain of the compounds described herein inhibit the DNA polymerases from both aerobic and anaerobic Gram-positive bacteria, they are in principle useful for inhibiting the growth of these bacteria. However, the results shown herein indicate selectivity for inhibiting the growth of C. diff. strains, sparing other Gram-positive anaerobic bacteria.

### Uses of the Compounds

Compounds of the invention can be used to inhibit growth of C. diff and to treating or reducing the likelihood of developing a C. diff-associated disease. The methods for inhibiting the growth of C. diff involve administering an effective amount of a compound of the invention. The degree of inhibition can be ascertained by an in vitro growth assay, e.g., by a standard liquid culture technique. Compounds showing inhibition of colony formation at suitable MICs (minimal inhibitory concentrations), e.g. <10 μg/ml, are useful for as therapeutic agents. The method for treating or reducing the likelihood of developing a C. diff.-associated infection involves administering a therapeutically effective amount of a compound of the invention, preferably by the oral route.

The compounds described herein are useful for the treatment of C. diff.-associated diseases, such as C. diff-associated diarrhea (CDAD) or colitis, including disease caused by highly virulent and antibiotic-resistant strains of C. diff. The compounds are useful for the treatment of such infections in humans and other animals, such as pigs, cows, horses, goats, chickens, turkeys, sheep, rats, mice, and rabbits.

In one embodiment, a compound or composition of the invention is administered to a subject that has been diagnosed with a C. diff.-associated infection. The compounds can be administered both prophylactically and after infection has occurred. Prophylaxis can be most appropriate for patients at risk for infection or for recurrence of an infection.

The compounds can be administered a subject susceptible to a C. diff. infection. Susceptibility to C. diff infection may occur as a result of antibiotic exposure, gastrointestinal surgery/manipulation, prolonged length of stay in a healthcare setting (e.g., greater than 1 week), serious underlying illness, immune-compromising conditions, aging, use of proton pump inhibitors, malignancy, chronic obstructive pulmonary disease, immunosuppressive or anti-peristaltic medications, inflammatory bowel disease, renal failure, hypoalbuminemia, and organ transplant. Peripartum women are also at increased risk of C. diff-associated infection. Antibiotics that may increase susceptibility to C. diff-associated infection include clindamycin, penicillins, cephalosporins, and fluoroquinolones.

Compounds of the invention may also be used in combination with other agents for treating or reducing the likelihood of developing C. diff-associated infections including vancomycin, metronidazole, and nitazoxanide.

This list of relevant conditions for application of the methods of the invention is not intended to be limiting, and any appropriate infection responsive to the compounds can be treated using the methods and/or compounds described herein.

### Pharmaceutical Compositions

The compounds of the invention may be formulated into pharmaceutical compositions for administration to human or animal subjects in a biologically compatible form suitable for administration in vivo or in vitro. Accordingly, the present invention provides a pharmaceutical composition including a compound of the invention in admixture with an excipient.

In accordance with the methods of the invention, the described compounds or salts thereof may be administered to a patient in a variety of forms depending on the selected route of administration, as will be understood by those skilled in the art. The compounds of the invention may be administered, for example, by oral, parenteral, buccal, sublingual, nasal, rectal, patch, pump, or transdermal administration and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time. For human or animal use, the formulations of this invention can be administered by the oral, buccal, rectal and vaginal routes, or by topical administration. The formulations of this invention may also be administered by the use of surgical implants which release the compounds of the invention, either as a bolus or slowly over a pre-selected period of time.

Without limitation, for oral administration, formulations can be, for example, in the form of tablets, capsules, liquid solutions and suspensions (wherein such solutions and suspensions are particularly for formulations intended for pediatric use).

A compound of the invention may also be administered parenterally. Solutions of a water-soluble compound of the invention can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19), published in 1999.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that it is easily administered via syringe.

Compositions for nasal administration may conveniently be formulated as aerosols, drops, gels, and powders. Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal after use. Where the dosage form comprises an aerosol dispenser, it will contain a propellant, which can be a compressed gas, such as compressed air or an organic propellant, such as fluorochlorohydrocarbon. The aerosol dosage forms can also take the form of a pump-atomizer.

Compositions suitable for buccal or sublingual administration include tablets, lozenges, and pastilles, where the active ingredient is formulated with a carrier, such as sugar, acacia, tragacanth, or gelatin and glycerine. Compositions for rectal administration are conveniently in the form of suppositories containing a conventional suppository base, such as cocoa butter.

The compounds of the invention may be administered to an animal, e.g., a human, alone or in combination with pharmaceutically acceptable excipients, as noted above, the proportion of which is determined by the solubility and chemical nature of the compound, chosen route of administration, and standard pharmaceutical practice.

The dosage of the compounds of the invention, and/or compositions comprising a compound of the invention, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds of the invention may be administered initially in a suitable dosage that may be adjusted as required, depending on the clinical response. In general, the compounds of the invention can be provided in an aqueous physiological buffer solution containing about 0.1 to 10% w/v compound or in a solid dosage form such as a tablet or capsule. General dose ranges are from about 0.01 mg/kg to about 1 g/kg of body weight per day.

### Water Solubility

The compounds of formula (I) generally have low water solubility; however, some of the compounds can form salts, such as with inorganic or organic acids, thus greatly increasing their water solubilities. The improved water solubilities are a distinct advantage in formulation and in dosing of animals for testing, and for ultimate therapeutic use in humans. Preferred pharmaceutically acceptable salts are hydrochloride salts. Other salts are described herein.

### General Synthetic Schemes

The preparation of compounds of the current application can be accomplished in a number of ways already well understood by those skilled in the art of organic synthesis. For example, the methods described below can be employed to synthesize compounds of the present application, together with other familiar synthetic methods in the art of synthetic organic chemistry or variations. A few preferred methods are described below among many others

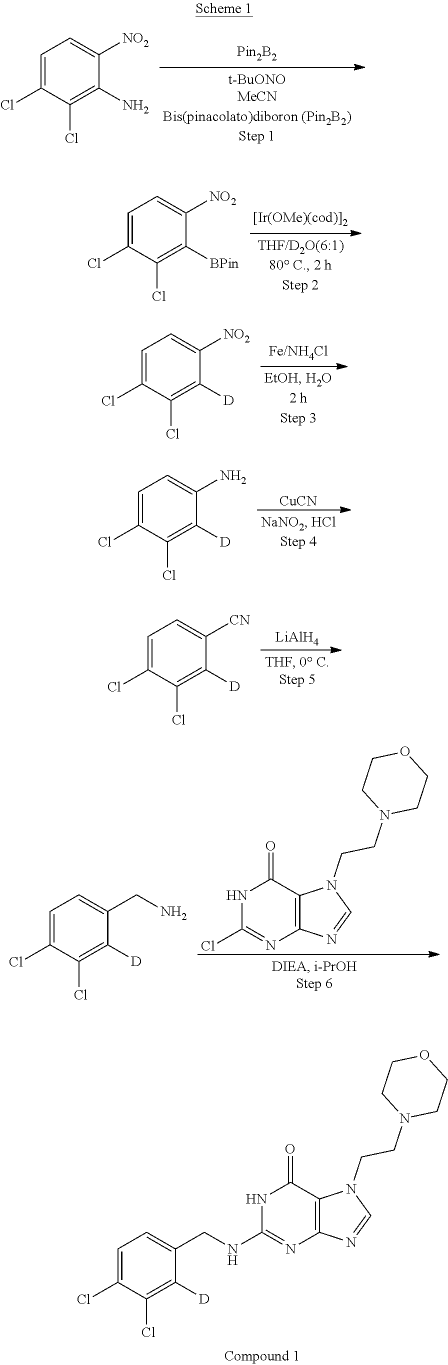

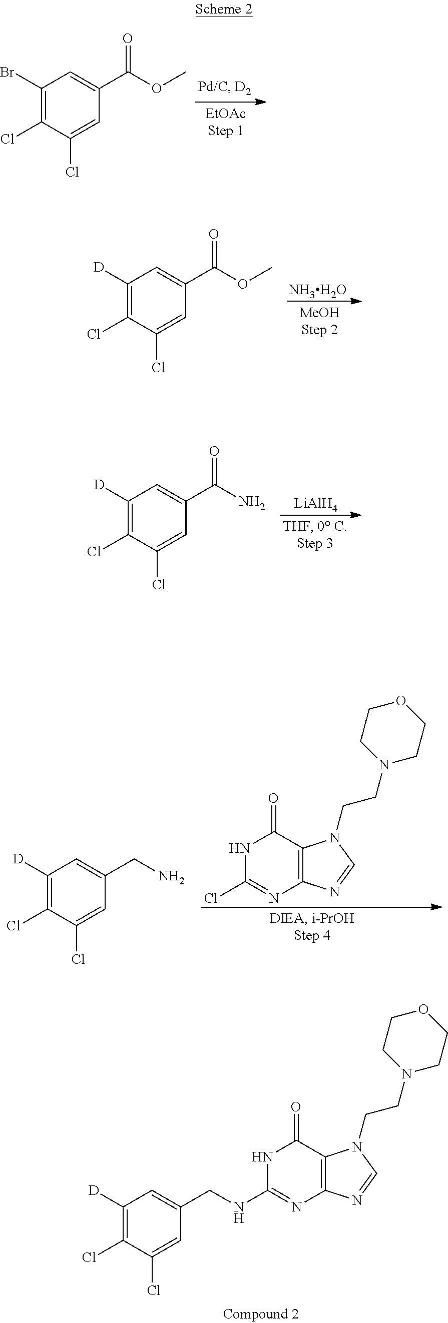

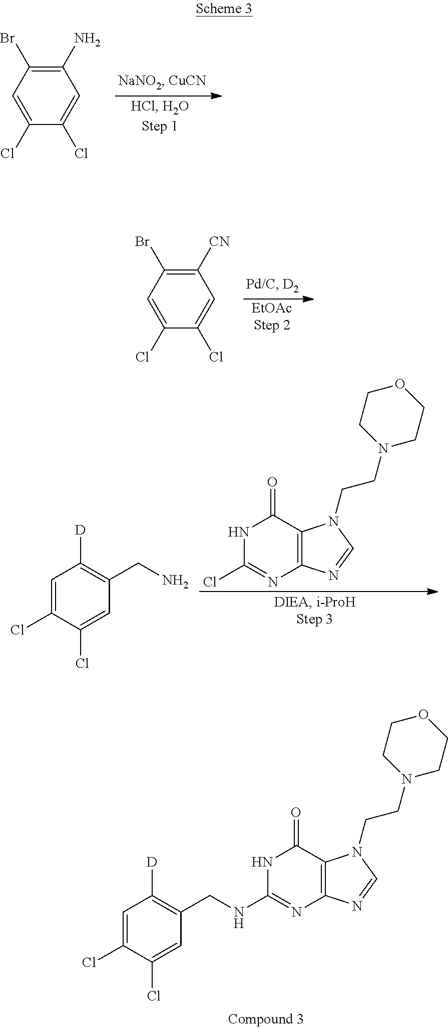 [SMILES: Nc1cc(Cl)c(Cl)cc1Br ; N#Cc1cc(Cl)c(Cl)cc1Br ; "EtOAcStep 2" ; [2H]c1cc(Cl)c(Cl)cc1CN ; O=c1[nH]c(Cl)nc2ncn(CCN3CCOCC3)c12 ; [2H]c1cc(Cl)c(Cl)cc1CNc1nc2ncn(CCN3CCOCC3)c2c(=O)[nH]1]

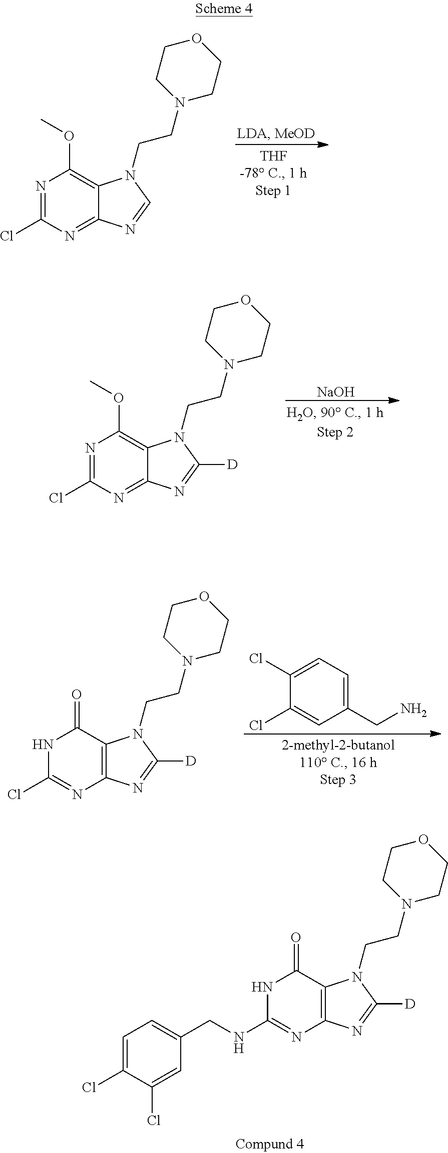

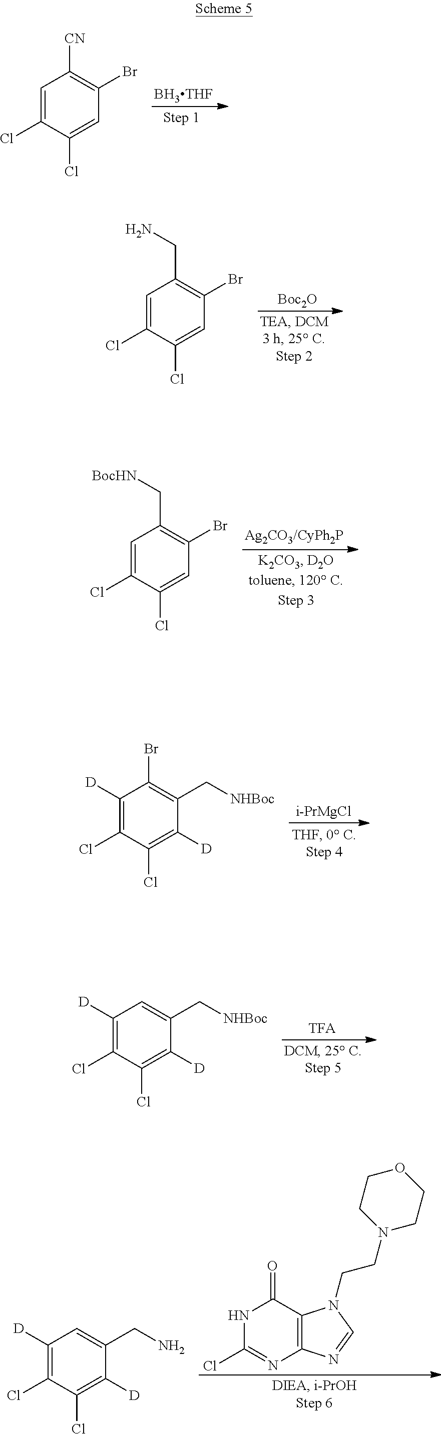

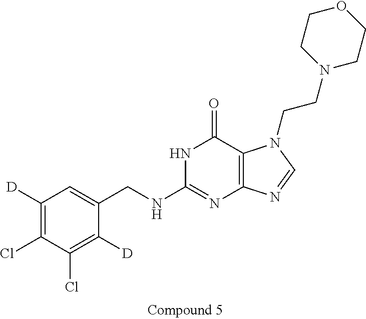 [SMILES: [2H]c1cc(CNc2nc3ncn(CCN4CCOCC4)c3c(=O)[nH]2)c([2H])c(Cl)c1Cl]

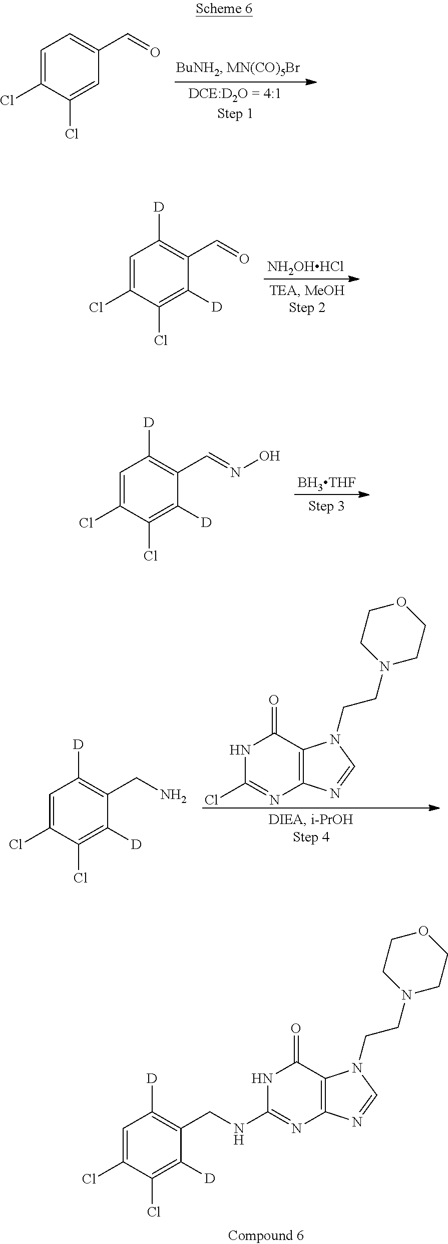

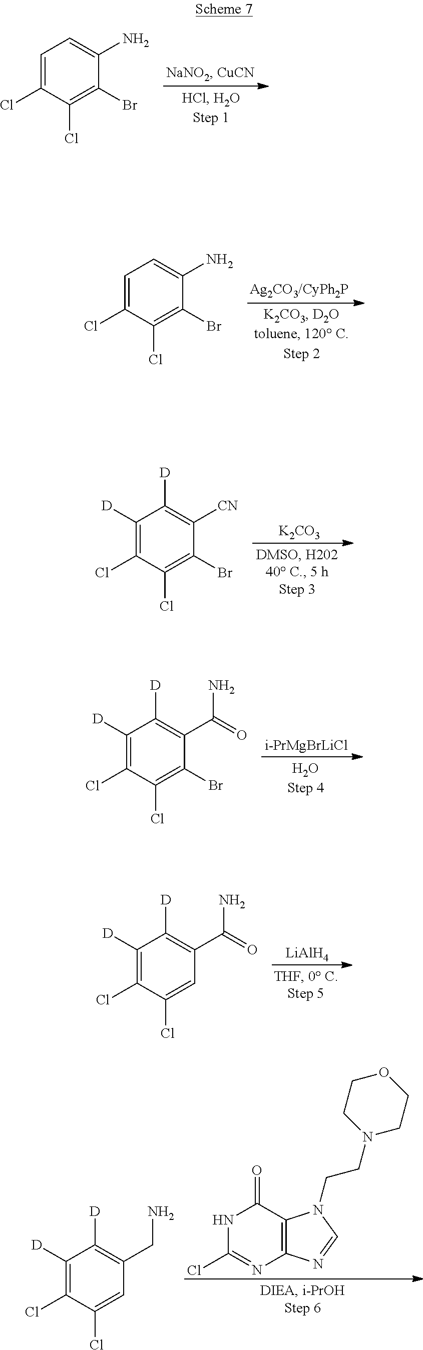

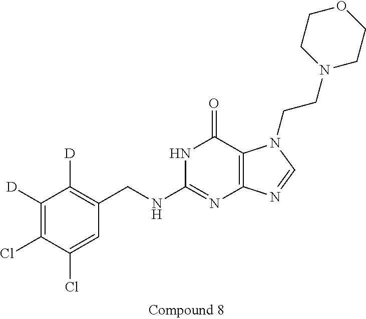 [SMILES: [2H]c1c(CNc2nc3ncn(CCN4CCOCC4)c3c(=O)[nH]2)cc(Cl)c(Cl)c1[2H]]

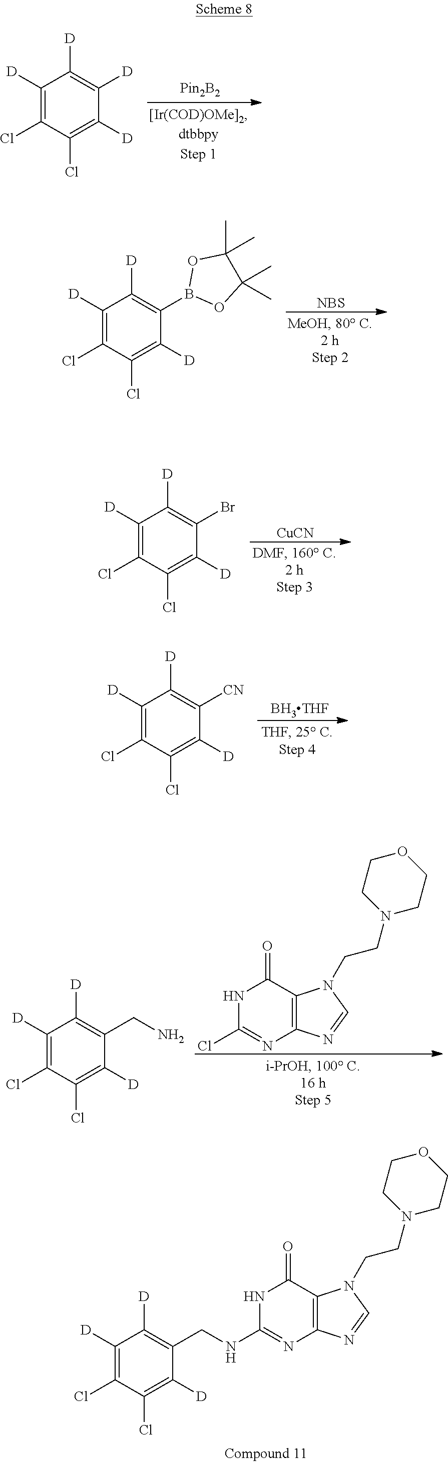

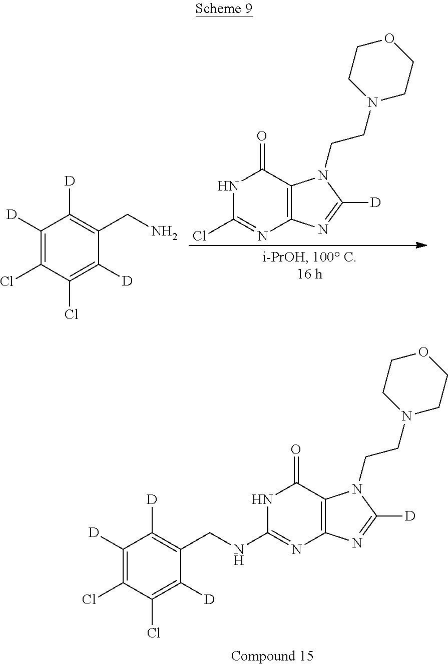 [SMILES: [2H]c1c([2H])c(CN)c([2H])c(Cl)c1Cl ; [2H]c1nc2nc(Cl)[nH]c(=O)c2n1CCN1CCOCC1 ; [2H]c1c([2H])c(CNc2nc3nc([2H])n(CCN4CCOCC4)c3c(=O)[nH]2)c([2H])c(Cl)c1Cl]

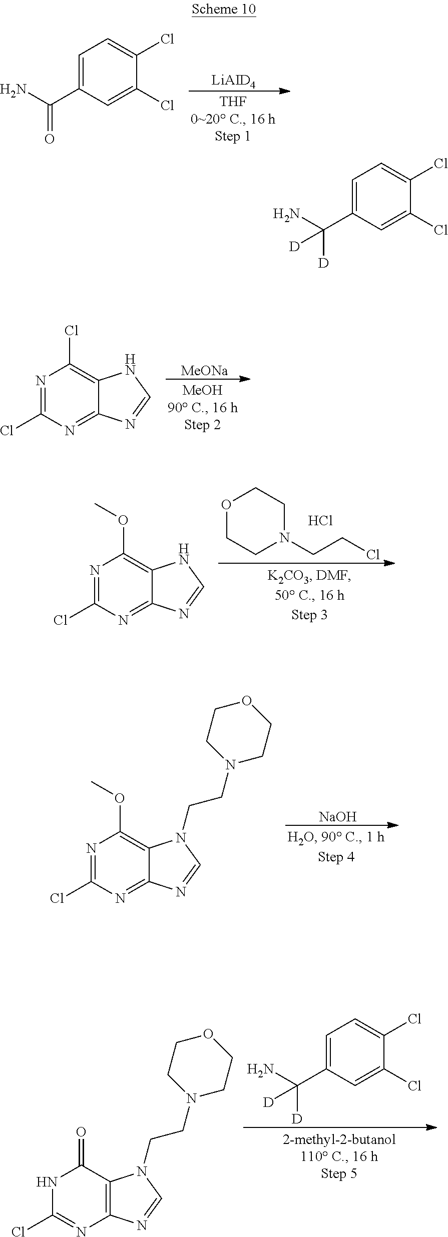

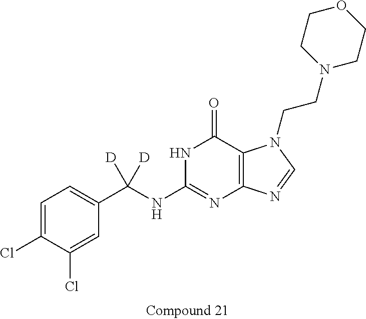 [SMILES: [2H]C([2H])(Nc1nc2ncn(CCN3CCOCC3)c2c(=O)[nH]1)c1ccc(Cl)c(Cl)c1]

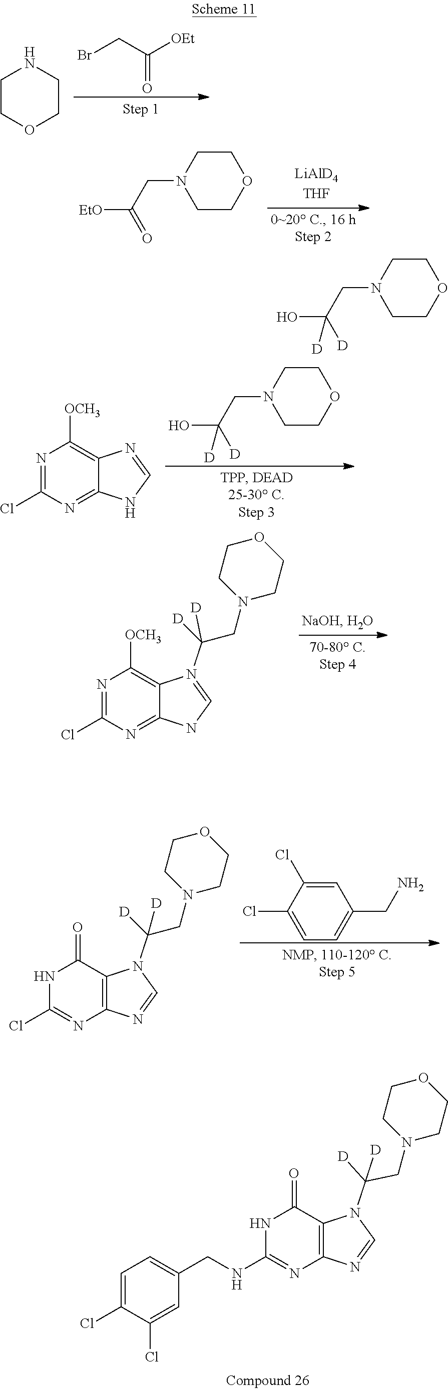

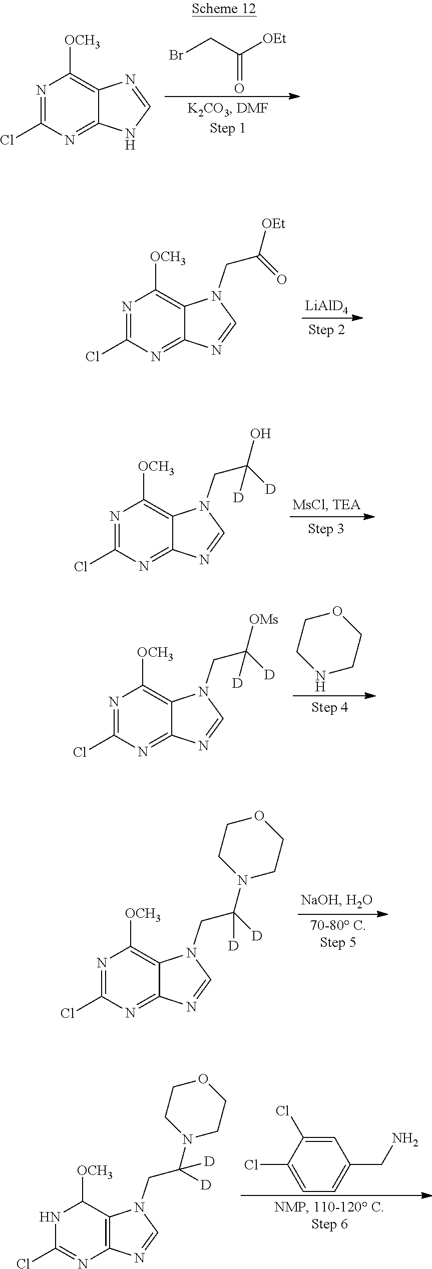

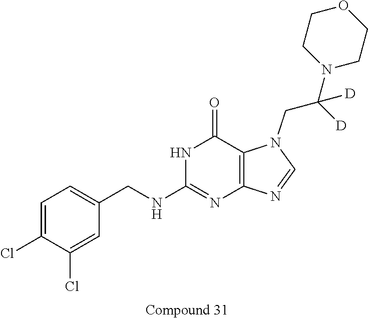 [SMILES: [2H]C([2H])(Cn1cnc2nc(NCc3ccc(Cl)c(Cl)c3)[nH]c(=O)c21)N1CCOCC1]

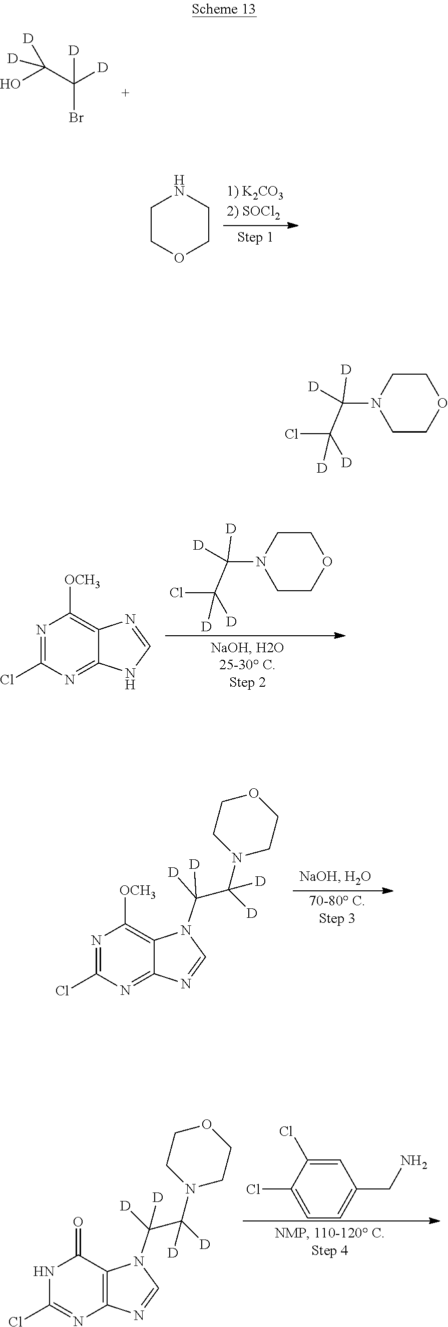

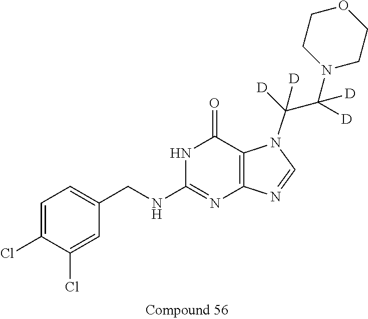 [SMILES: [2H]C([2H])(N1CCOCC1)C([2H])([2H])n1cnc2nc(NCc3ccc(Cl)c(Cl)c3)[nH]c(=O)c21]

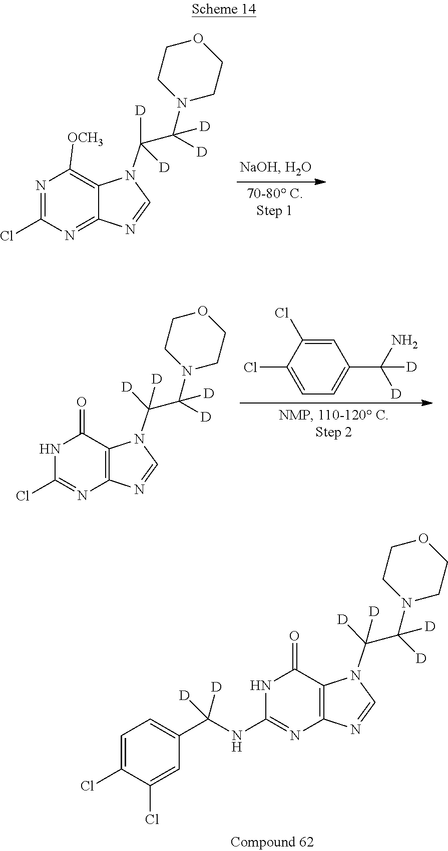

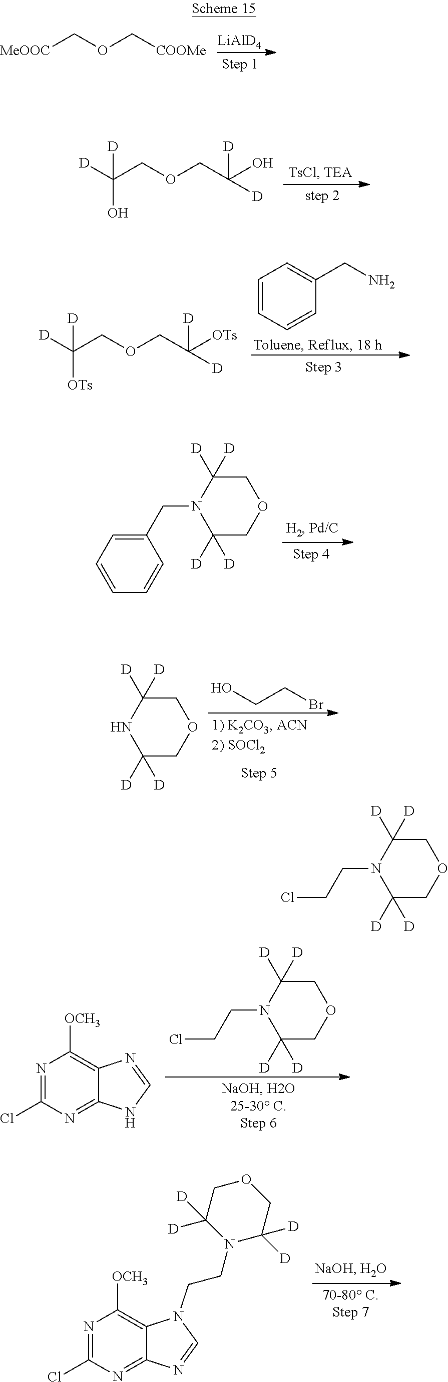

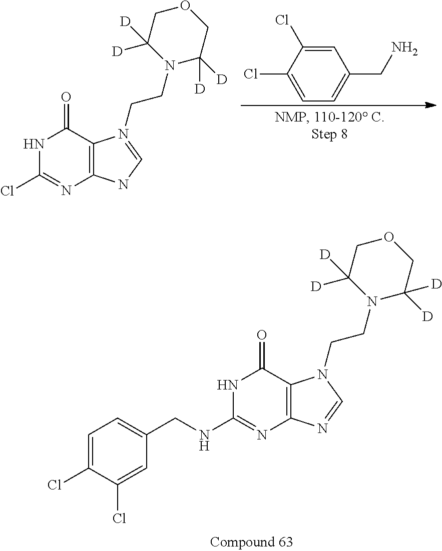

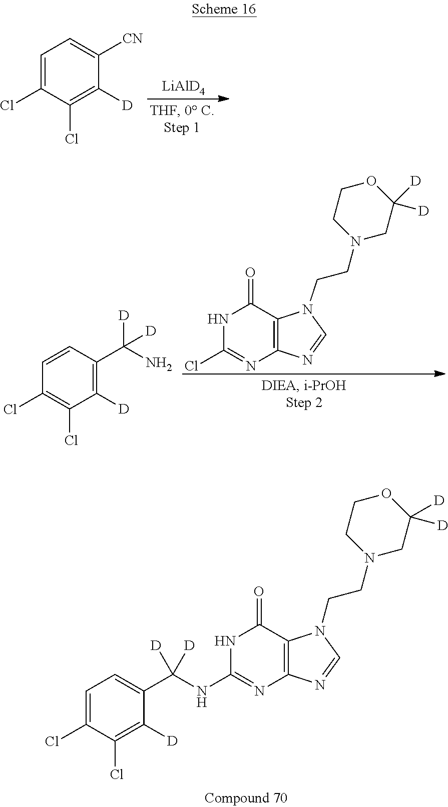

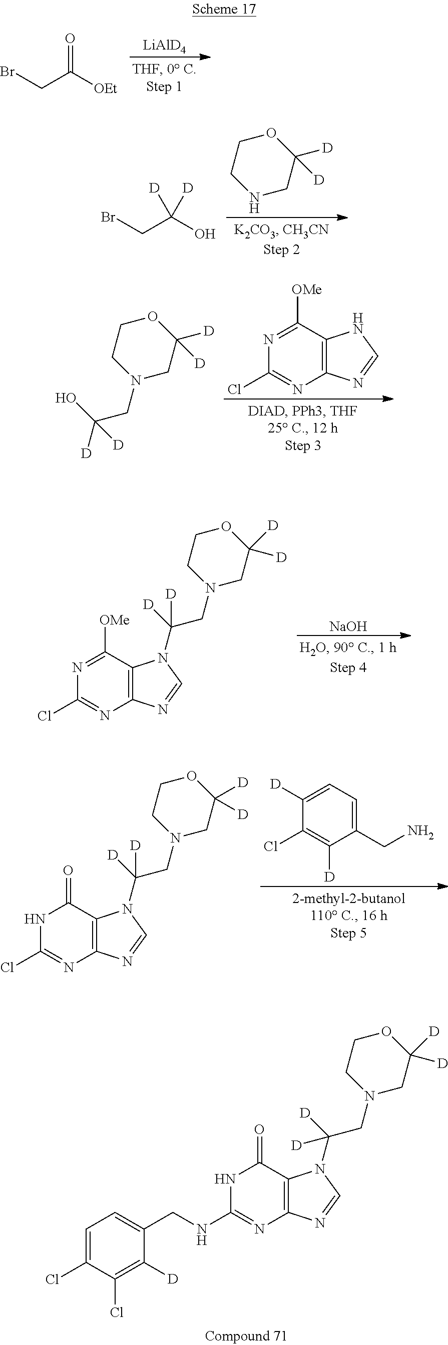

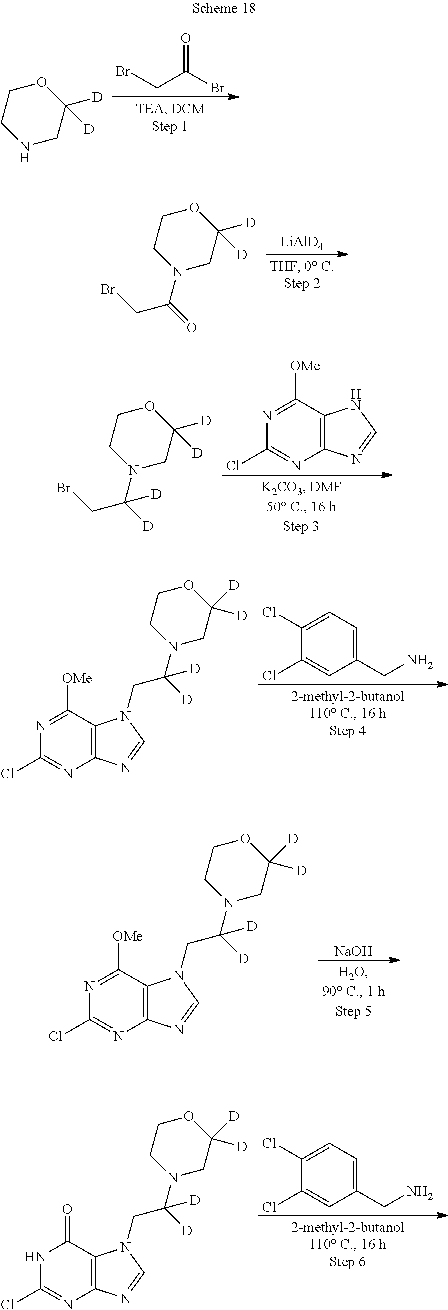

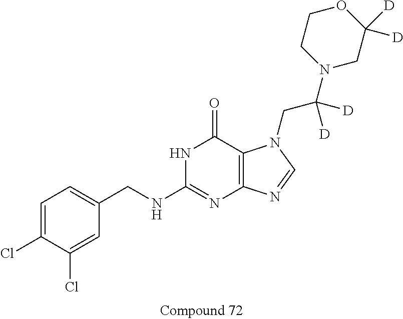 [SMILES: [2H]C1([2H])CN(C([2H])([2H])Cn2cnc3nc(NCc4ccc(Cl)c(Cl)c4)[nH]c(=O)c32)CCO1]

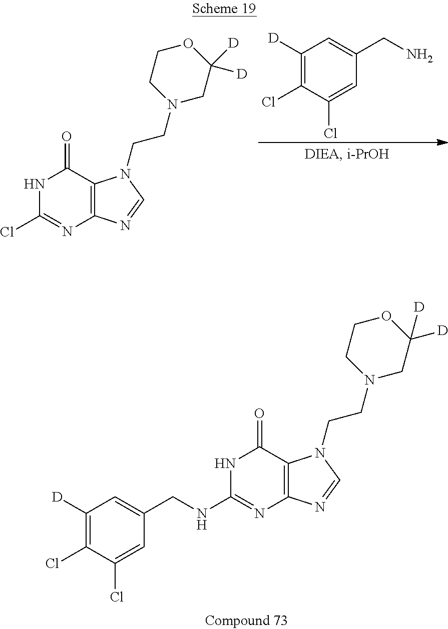

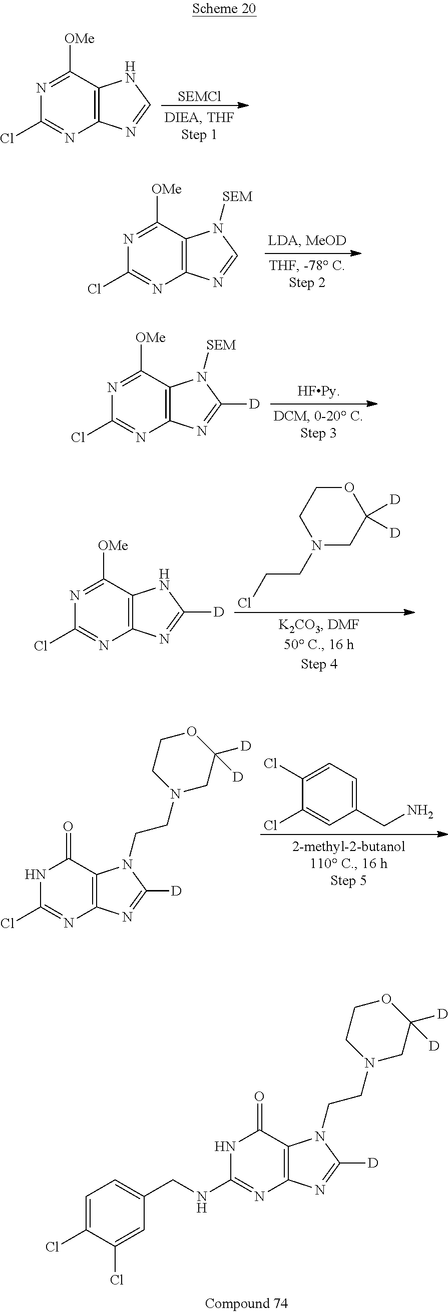

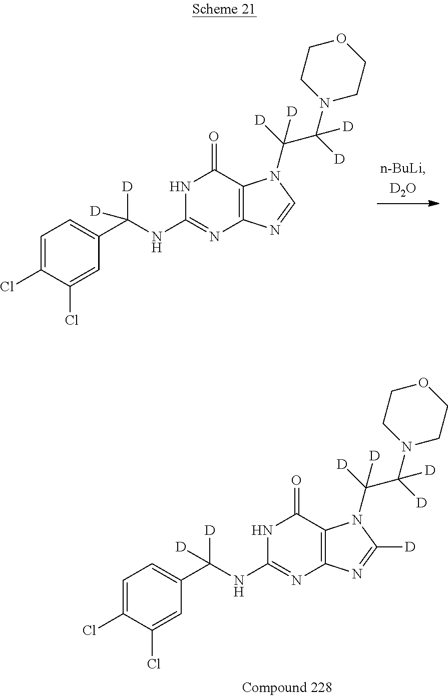 [SMILES: [2H]C([2H])(Nc1nc2ncn(C([2H])([2H])C([2H])([2H])N3CCOCC3)c2c(=O)[nH]1)c1ccc(Cl)c(Cl)c1 ; [2H]c1nc2nc(NC([2H])([2H])c3ccc(Cl)c(Cl)c3)[nH]c(=O)c2n1C([2H])([2H])C([2H])([2H])N1CCOCC1]

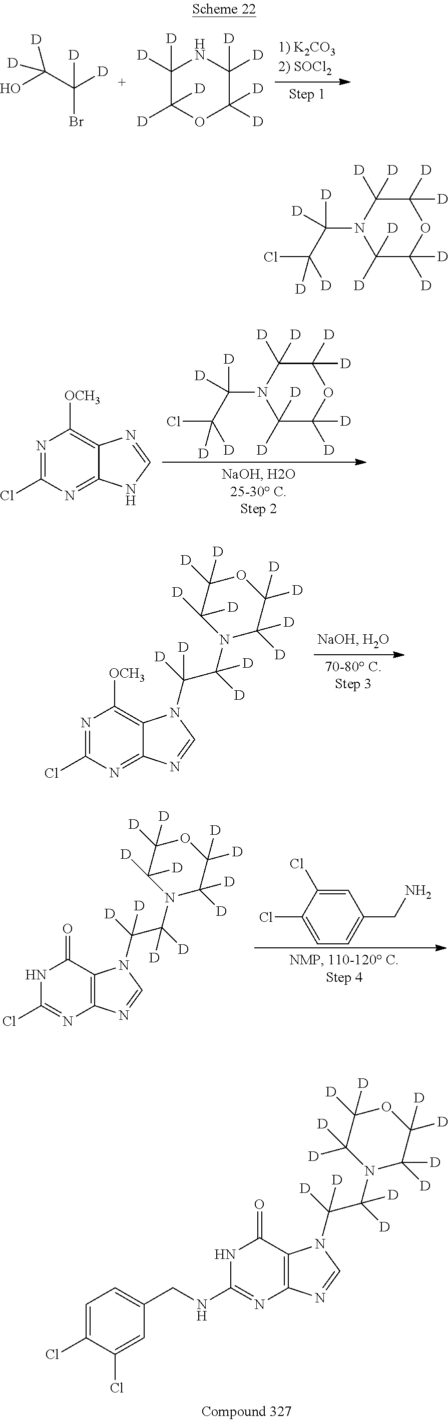

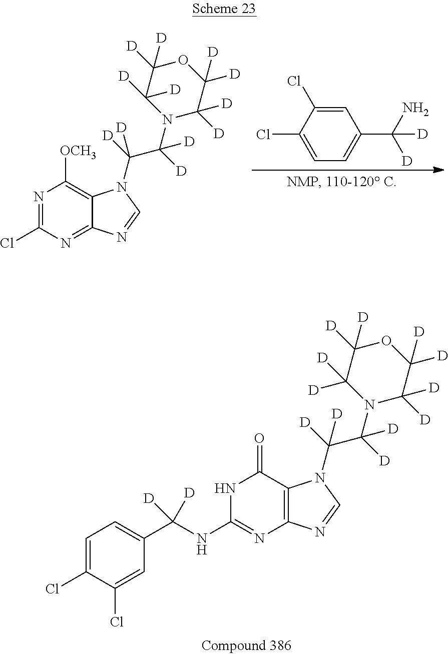

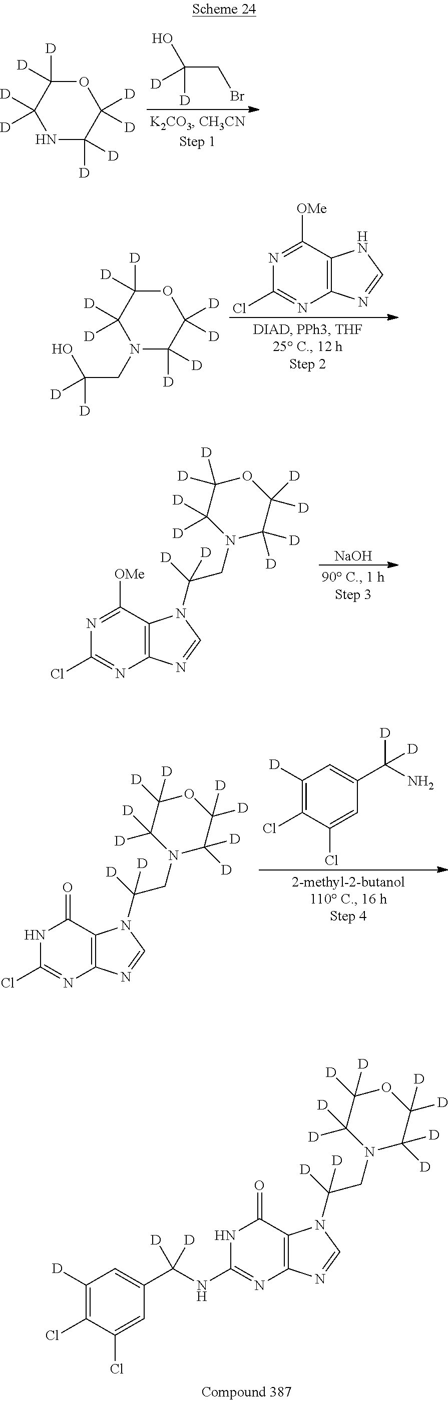

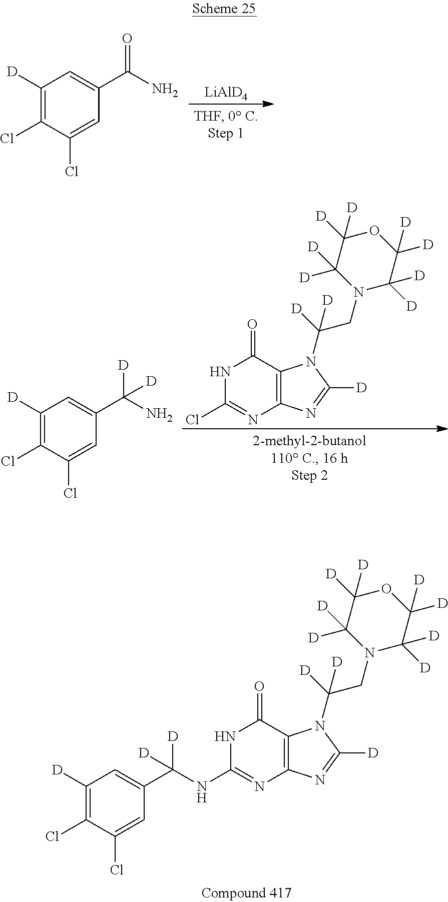

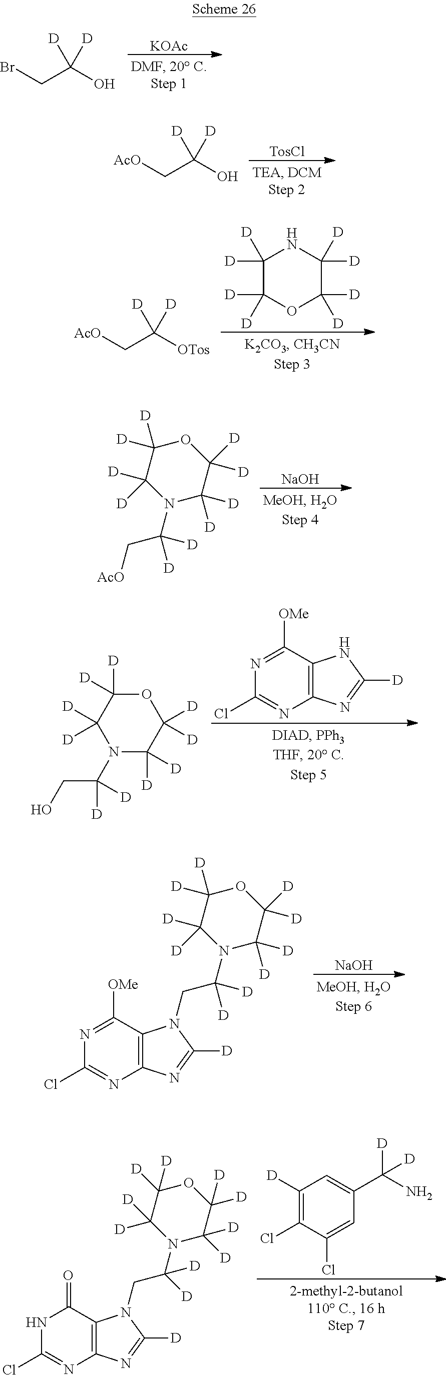

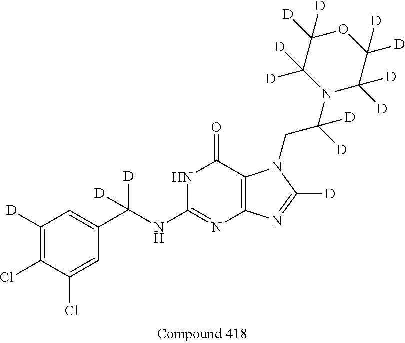 [SMILES: [2H]c1cc(C([2H])([2H])Nc2nc3nc([2H])n(CC([2H])([2H])N4C([2H])([2H])C([2H])([2H])OC([2H])([2H])C4([2H])[2H])c3c(=O)[nH]2)cc(Cl)c1Cl]

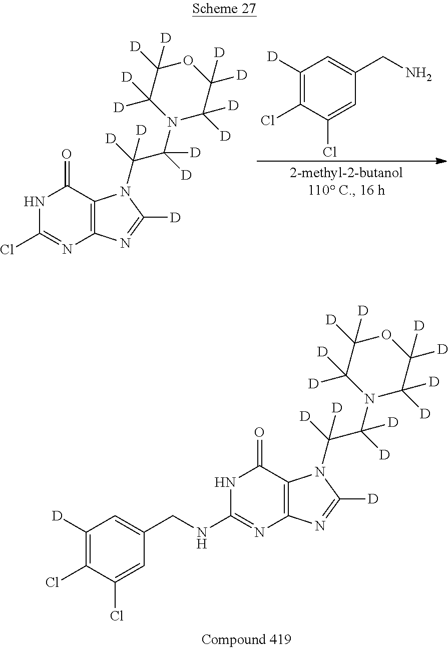

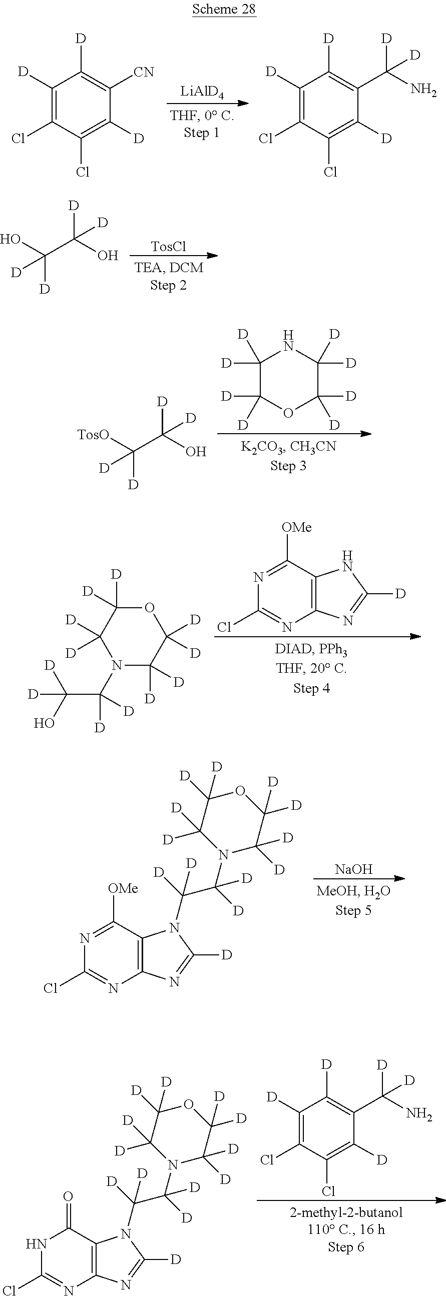

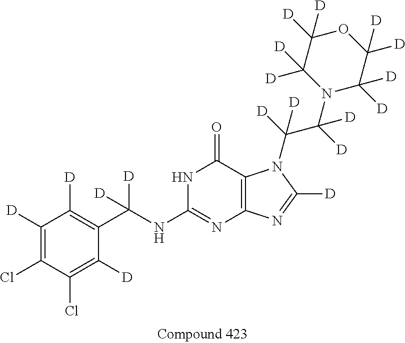 [SMILES: [2H]c1c([2H])c(C([2H])([2H])Nc2nc3nc([2H])n(C([2H])([2H])C([2H])([2H])N4C([2H])([2H])C([2H])([2H])OC([2H])([2H])C4([2H])[2H])c3c(=O)[nH]2)c([2H])c(Cl)c1Cl]

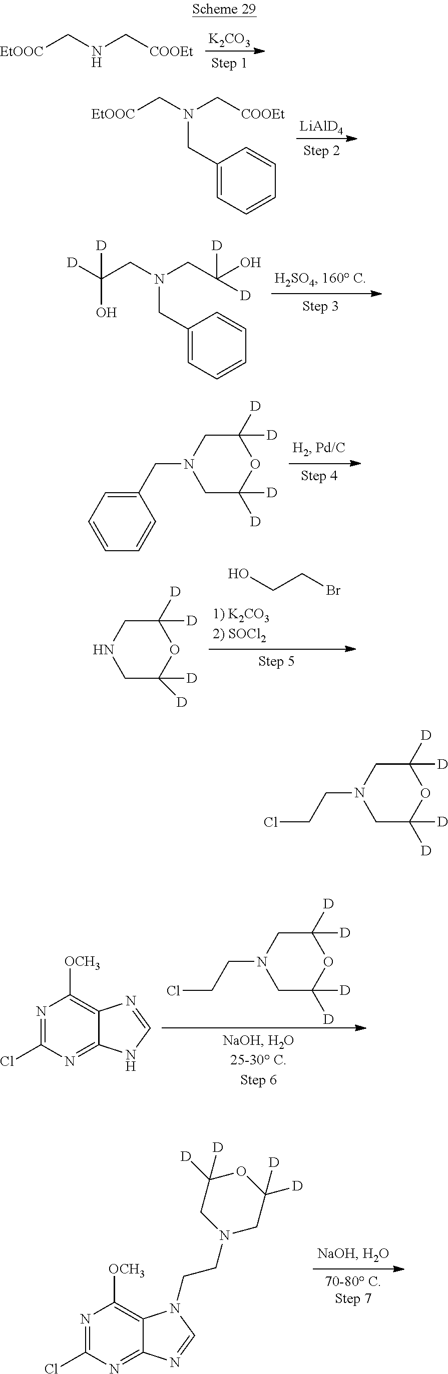

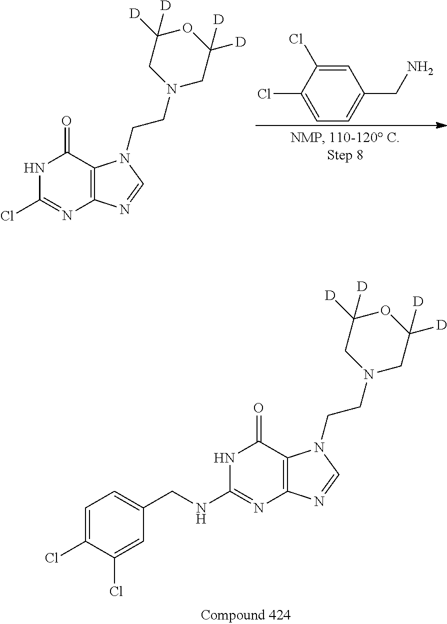 [SMILES: [2H]C1([2H])CN(CCn2cnc3nc(Cl)[nH]c(=O)c32)CC([2H])([2H])O1 ; NCc1ccc(Cl)c(Cl)c1 ; [2H]C1([2H])CN(CCn2cnc3nc(NCc4ccc(Cl)c(Cl)c4)[nH]c(=O)c32)CC([2H])([2H])O1]

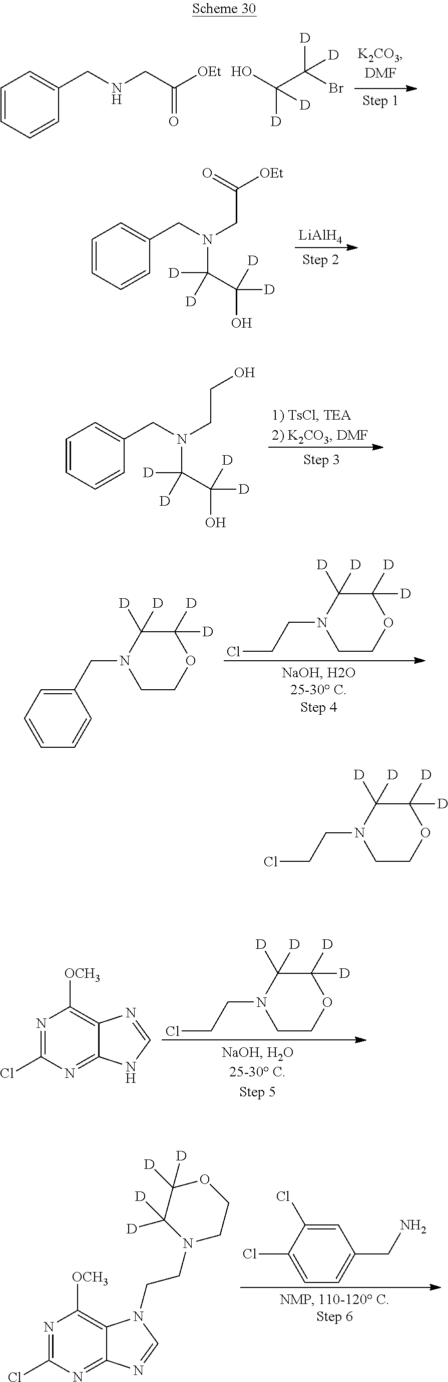

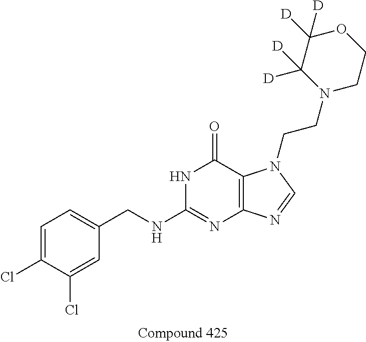 [SMILES: [2H]C1([2H])OCCN(CCn2cnc3nc(NCc4ccc(Cl)c(Cl)c4)[nH]c(=O)c32)C1([2H])[2H]]

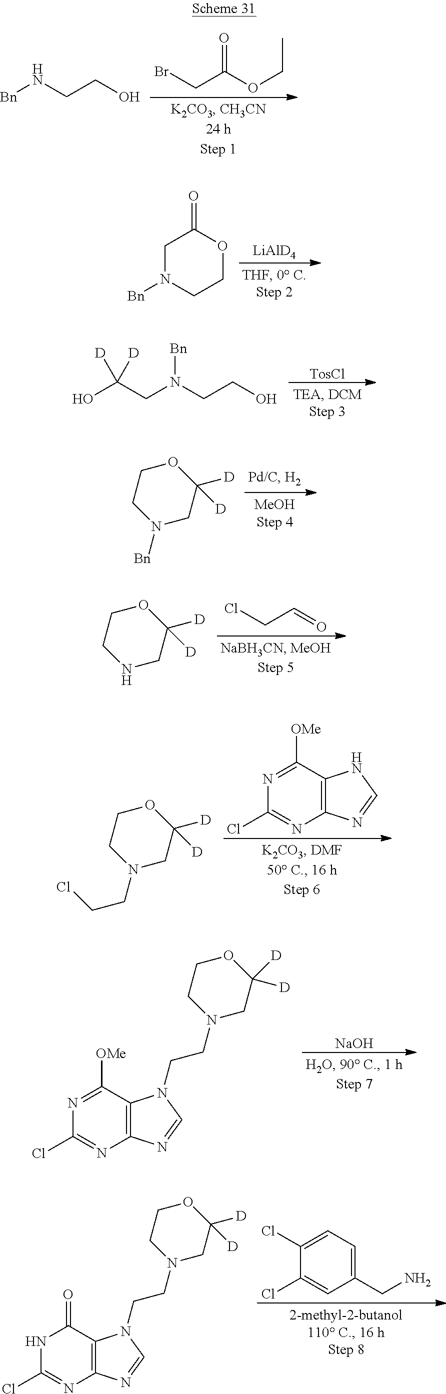

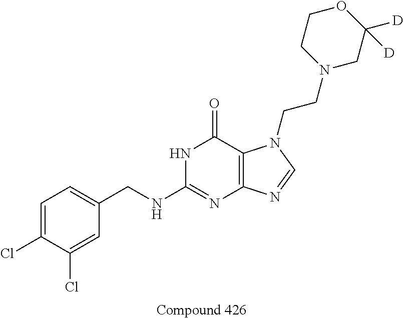 [SMILES: [2H]C1([2H])CN(CCn2cnc3nc(NCc4ccc(Cl)c(Cl)c4)[nH]c(=O)c32)CCO1]

### EXAMPLE

### Preparation of Compound 21

### 2-[[dideuterio-(3,4-dichlorophenyl)methyl]amino]-7-(2-morpholinoethyl)-1H-purin-6-one (Steps 1-5 in Scheme 10)

### Preparation of dideuterio-(3,4-dichlorophenyl) methanamine (Step 1 in Scheme 10)

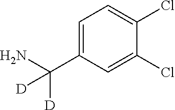 [SMILES: [2H]C([2H])(N)c1ccc(Cl)c(Cl)c1]

To a solution of 3,4-dichlorobenzamide (200 mg, 1.05 mmol, 1 eq) in THF (5 mL) was added lithium tetradeuterioalumanuide (119.84 mg, 3.16 mmol, 162.82 μL, 3 eq) at 0° C. Then the mixture was stirred at 20° C. for 16 hrs. LC-MS showed the reaction was complete. The reaction mixture was quenched with D20 (2 mL) at 0° C. and diluted with H₂O (20 mL). The organic solvent was removed under reduced pressure. The aqueous was extracted with EtOAc (10 mL×3). The combined organic layers were washed with brine (10 mL), dried over Na₂SO₄, filtered and concentrated under reduced pressure. The residue was purified by flash silica gel chromatography (Biotage®; 4 g SepaFlash® Silica Flash Column, eluent of 0-10% ethyl acetate/MeOH gradient @ 30 mL/min). Compound dideuterio-(3,4-dichlorophenyl) methanamine (100 mg, 561.62 μmol, 53.36% yield) was obtained as yellow oil. ¹H NMR (CDCl₃, 400 MHz) δ 7.43 (d, J=2.0 Hz, 1H), 7.40 (d, J=8.0 Hz, 1H), 7.16 (dd, J=8.4 Hz, 2.0 Hz, 1H).

### Preparation of 2-chloro-6-methoxy-9H-purine (Step 2 in Scheme 10)

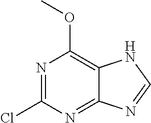

To a solution of 2,6-dichloro-9H-purine (15 g, 79.36 mmol, 1 eq) in MeOH (20 mL) was added NaOMe (4.72 g, 87.30 mmol, 1.1 eq) at 20° C. Then the mixture was stirred at 80° C. for 1 hour. TLC (petroleum ether:ethyl acetate=0:1) showed the reaction was complete. The reaction was cooled to 20° C. Then the mixture was acidified to pH=7 with AcOH. The mixture was concentrated. Water (450 mL) was added and stirred for 10 min. The mixture was filtered and the filter cake was washed with water (100 mL×2). The crude product was purified by trituration from MeOH (50 mL) at 20° C. Compound 2-chloro-6-methoxy-9H-purine (10 g, 54.18 mmol, 68.26% yield) was obtained as white solid. ¹H NMR (DMSO-d6, 400 MHz) δ 8.39 (s, 1H), 4.08 (s, 3H).

### Preparation of 4-[2-(2-chloro-6-methoxy-purin-7-yl) ethyl]morpholine (Step 3 in Scheme 10)

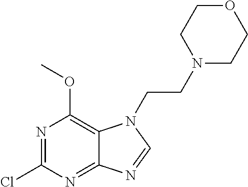 [SMILES: COc1nc(Cl)nc2ncn(CCN3CCOCC3)c12]

To a solution of 2-chloro-6-methoxy-9H-purine (5 g, 27.09 mmol, 1 eq) and 4-(2-chloroethyl)morpholine (7.56 g, 40.63 mmol, 1.5 eq, HCl) in DMF (50 mL) was added K₂CO₃ (11.23 g, 81.26 mmol, 3 eq) at 20° C. Then the mixture was stirred at 50° C. for 16 hours. LCMS showed the reaction was complete. The residue was poured into ice-water (200 mL) and stirred for 10 min. The aqueous phase was extracted with ethyl acetate (100 mL×3). The combined organic phase was washed with brine (100 mL×2), dried with anhydrous Na₂SO₄, filtered and concentrated in vacuum. The residue was purified by flash silica gel chromatography (Biotage®; 120 g SepaFlash® Silica Flash Column, eluent of 0-15% ethyl acetate/MeOH gradient @ 100 mL/min). Compound 4-[2-(2-chloro-6-methoxy-purin-7-yl) ethyl]morpholine (2 g, 6.72 mmol, 24.80% yield) was obtained as white solid. ¹H NMR (DMSO-d6, 400 MHz) δ 8.51 (s, 1H), 4.40 (t, J=6.0 Hz, 2H), 4.10 (s, 3H) 3.50 (t, J=4.4 Hz, 4H), 2.67 (t, J=6.0 Hz, 2H), 2.40 (t, J=4.4 Hz, 2H).

### Preparation of 2-chloro-7-(2-morpholinoethyl)-1H-purin-6-one (Step 4 in Scheme 10)

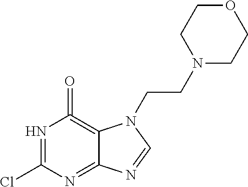

To a solution of 4-[2-(2-chloro-6-methoxy-purin-7-yl)ethyl]morpholine (1 g, 3.36 mmol, 1 eq) in H₂O (1 mL) was added NaOH (1 M, 5.04 mL, 1.5 eq) at 20° C. Then the mixture was stirred at 80° C. for 1 hrs. LC-MS showed the reaction was complete. The reaction was cooled to 20° C. Then the mixture was acidified to pH=7 with HCl (6 M). The residue was purified by prep-HPLC (column: Phenomenex Luna C18 250 mm*100 mm*10 um; mobile phase: [water (10 mM NH₄HCO₃)-ACN]; B %: 0%-15%, 25 min). The eluent was removed and freeze dried to give product. Compound 2-chloro-7-(2-morpholinoethyl)-1H-purin-6-one (500 mg, 1.76 mmol, 52.47% yield) was obtained as white solid. ¹H NMR (DMSO-d6, 400 MHz) δ 8.21 (s, 1H), 4.40 (t, J=6.0 Hz, 2H), 3.50 (t, J=4.4 Hz, 4H), 2.69 (t, J=6.0 Hz, 2H), 2.40 (t, J=4.4 Hz, 2H).

### Preparation of 2-[[dideuterio-(3,4-dichlorophenyl)methyl]amino]-7-(2-morpholinoethyl)-1H-purin-6-one (Step 5 in Scheme 10)

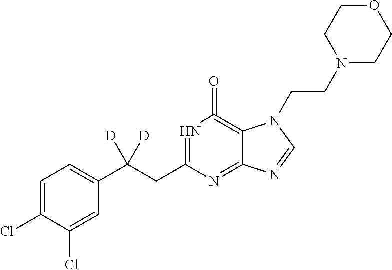

To a solution of 2-chloro-7-(2-morpholinoethyl)-1H-purin-6-one (50 mg, 176.23 μmol, 1 eq) in 2-methyl-2-buanol (1 mL) was added dideuterio-(3,4-ichlorophenyl)methanamine (47.07 mg, 264.35 μmol, 1.5 eq) at 20° C. Then the mixture was stirred at 110° C. for 16 hrs. LC-MS showed 2-chloro-7-(2-morpholinoethyl)-1H-purin-6-one was consumed completely and one main peak with desired mass was detected. The reaction was cooled to 20° C. Then the mixture was concentrated under reduced pressure at 40° C. The residue was purified by prep-HPLC (column: Waters Xbridge BEH C18 100*25 mm*5 um; mobile phase: [water (10 mM NH₄HCO₃)-ACM; B %: 20%-45%, 10 min). The eluent was removed and freeze dried to give product. Compound 2-[[dideuterio-(3,4-dichlorophenyl)methyl]amino]-7-(2-morpholinoethyl)-1H-purin-6-one (25.7 mg, 59.25 μmol, 33.62% yield, 98.06% purity) was obtained as white solid. ¹H NMR (DMSO-d6, 400 MHz) δ 7.91 (s, 1H), 7.62-7.55 (m, 2H), 7.33 (dd, J=8.0 Hz, 2.0 Hz, 1H), 6.62 (s, 1H), 4.28 (t, J=6.0 Hz, 2H), 3.50 (t, J=4.4 Hz, 4H), 2.65 (t, J=6.0 Hz, 2H), 2.38 (t, J=4.4 Hz, 2H). HPLC: 98.06% (220 nm), 98.83% (254 nm). MS (ESI): mass calcd. For C₁₈H₁₈D₂Cl₂N₆O₂ 424.12 m/z found 425.0 [M+H]⁺.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

A number of embodiments of the invention have been described. Nevertheless, it will be understood that various modifications can be made without departing from the spirit and scope of the invention. Accordingly, other embodiments are within the scope of the following claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

### REFERENCES

Wright, et al., “Inhibition of Bacillus subtilis DNA Polymerase III by Arylhydrazinopyrimidine” Biochimica et Biophysica Acta 1976, 432, 37-48
Tarantino, et al., “6-Anilinouracil-Based Inhibitors of Bacillus subtilis DNA Polymerase III: Antipolymerase and Antimicrobial Structure-Activity Relationships Based on Substitution at Uracil N3” J. Med. Chem. 1999, 42, 2035-2040.
Tarantino, et al., “Inhibitors of DNA polymerase III as novel antimicrobial agents against Gram-positive eubacteria” Antimicr. Agents Chemother. 1999, 43, 1982-1987.
Daly, et al., “In vitro antimicrobial activity of novel anilinouracils which selectively inhibit DNA polymerase III of Gram-positive bacteria” Antimicr. Agents Chemother. 2000, 44, 2217-2221.
Ali, et al., “Design and Synthesis of Novel Antibacterial Agents with Inhibitory Activity against DNA Polymerase III” Bioorg. Med. Chem. Lett. 2001, 11, 2185-2188.
Muto, et al., “SHEA Guideline for Preventing Nosocomial Transmission of Multidrug-Resistant Strains of Staphylococcus aureus and Enterococcus” Infection Control and Hospital Epidemiology 2003, 362-386.
Ali, et al., “Novel Pyrazolo [3,4-d]pyrimidine-Based Inhibitors of Staphylococcus aureus DNA Polymerase III: Design, Synthesis, and Biological Evaluation” J. Med. Chem. 2003, 46, 1824-1830.
Zhi, et al., “Synthesis and Antibacterial Activity of 3-Substituted-6-(3-ethyl-4-methylanilino) uracils” J. Med. Chem. 2005, 48, 7063-7074.
Wright. et al., “Active site directed inhibitors of replication-specific bacterial DNA polymerases” Bioorg Med Chem Lett. 2005, 15, 729-732.
Kuhl, et al., “Biological Characterization of Novel Inhibitors of the Gram-Positive DNA Polymerase IIIC Enzyme” Antimicr. Agents Chemother. 2005, 49, 987-995.
Wright, et al., “Purine and Isosteric Antibacterial Compounds” U.S. Pat. No. 6,926,763 (2005)
Zhi, et al., “Hybrid Antibacterials. DNA polymerase topoisomerase inhibitors.” J. Med. Chem. 2006, 49, 1455-1465.
Evans, et al., “Structure of Po/C reveals unique DNA binding and fidelity determinants” Proc Natl Acad Sci USA 2008, 105, 20695-700.
Cer, et al., “IC₅₀-to-Ki: a web-based tool for converting IC₅₀ to Ki values for inhibitors of enzyme activity and ligand binding” Nucleic Acids Research, 2009, Vol. 37, Web Server issue W441-W445 doi:10.1093/nar/gkp253
Hookman. et al., “Clostridium difficile associated infection, diarrhea and colitis,” World J. Gastroenterol 2009, 15, 1554-1580.
Torti, et al., “Clostridium difficile DNA polymerase IIIC: Basis for activity of Anti-Bacterial Compounds” Current Enzyme Inhibition 2011, 7, 147-153.
Xu, et al., “7-Alkyl-N2-substituted-3-deazaguanines. Synthesis, DNA polymerase III inhibition and antibacterial activity” Bioorg Med Chem Lett. 2011, 21, 4197-202.
Dvoskin, et al., “A novel agent effective against infection with Clostridium difficile” Antimicr. Agents Chemother. 2012, 56, 1624-1626.
Guile, et al., “Antibacterial sulfone and sulfoxide substituted heterocyclic urea compounds” U.S. Pat. No. 8,293,919 (2012),
Guile, et al., “Antibacterial heterocyclic ureas” U.S. Pat. No. 8,716,320 (2014).
Wright, et al., “Selective Antibacterials for Clostridium difficile Infections” U.S. Pat. No. 8,796,292 (2014).
Smits, et al “Clostridium difficile infection. Nature Rev” 2: 1-20, Wilcox M H, Kuiiper E J (2016).
McDonald, et al. “Clinical Practice Guidelines for Clostridium difficile Infection in Adults and Children: 2017 Update by the Infectious Diseases Society of America (IDSA) and Society for Healthcare Epidemiology of America (SHEA).” Clin Infect Dis 2018. 66(7): 987-94.
Magill, et al., “Changes in Prevalence of Health Care—Associated Infections in U.S. Hospitals” The New England Journal of Medicine 2018, 379, 1732-1744.
van Eijk, et al “Genome Location Dictates the Transcriptional Response to PolC Inhibition in Clostridium difficile.” Antimicrob Agents Chemother 2019; 63(2).
“WHO (2019) Antibacterial agents in clinical development: an analysis of the antibacterial clinical development pipeline. Geneva: World Health Organization”, 2019.
Xu, et al “Discovery and development of DNA polymerase IIIC inhibitors to treat Gram-positive infections” Bioorg. Med. Chem. Vol. 27, Issue 15, 3209-3217 (2019).
Murray, et al “In vitro activity of the novel antibacterial agent ibezapolstat against Clostridioides difficile.” J Antimicrob Chemother. doi:10.1093/jac/dkaa134, (2020).
Normington, et al “Biofilms harbour clostridioides difficile, serving as a reservoir for recurrent infection. Accepted for publication: Npj Biofilms and Microbiomes (NPJBIOFILMS-00683R), Wilcox M H, Buckley A M (2020, in press)
Yu, et al “DNA Polymerase IIIC Inhibitors and Use Thereof” U.S. Pat. No. 10,723,741 (2020).

What is claimed is:

1. A compound represented by Formula I: 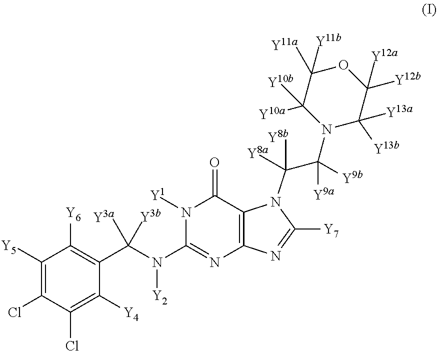
or a pharmaceutically acceptable salt thereof, wherein:
each of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} is independently selected from hydrogen or deuterium; and
at least one of Y¹, Y², Y^{3a}, Y^{3b}, Y⁴, Y⁵, Y⁶, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} is deuterium;
The invention excludes the compound with Y¹, Y², Y^{3a}, Y^{3b}, Y⁴, Y⁵, Y⁶, Y⁷, Y^{8a}, Y^{8b}, Y^{9a}, Y^{9b} as hydrogen and Y^{10a}, Y^{10b}, Y^{11a}, Y^{11b}, Y^{12a}, Y^{12b}, Y^{13a} and Y^{13b} as deuterium.

2. The compound of claim 1, wherein at least one of Y⁴, Y⁵, Y⁶, and Y⁷ is deuterium.

3. The compound of claim 2, wherein the compound is selected from any one of the compounds set forth in the table below:

| Compound | Y4 | Y5 | Y6 | Y⁷ |
|---|---|---|---|---|
| 1 | D | H | H | H |
| 2 | H | D | H | H |
| 3 | H | H | D | H |
| 4 | H | H | H | D |
| 5 | D | D | H | H |
| 6 | D | H | D | H |
| 7 | D | H | H | D |
| 8 | H | D | D | H |
| 9 | H | D | H | D |
| 10 | H | H | D | D |
| 11 | D | D | D | H |
| 12 | D | D | H | D |
| 13 | D | H | D | D |
| 14 | H | D | D | D |
| 15 | D | D | D | D |

wherein Y¹, Y², Y^{3a}, Y^{3b}, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} are all H.

4. The compound of claim 2, wherein the level of deuterium incorporation at each of Y⁴, Y⁵, Y⁶, and Y⁷ designated as deuterium is at least 52.5%, at least 75%, at least 82.5%, at least 90%, at least 95%, at least 97%, at least 98%, or at least 99%.

5. The compound of claim 1, wherein at least one of least one of Y^{3a}, Y^{3b}, Y^{8a}, Y^{8b}, Y^{9a} and Y^{9b} is deuterium.

6. The compound of claim 5, wherein the compound is selected from any one of the compounds set forth in the table below:

| Compound | Y^{3a} | Y^{3b} | Y^{8a} | Y^{8b} | Y^{9a} | Y^{9b} |
|---|---|---|---|---|---|---|
| 16 | D | H | H | H | H | H |
| 17 | H | H | D | H | H | H |
| 18 | H | H | H | D | H | H |
| 19 | H | H | H | H | D | H |
| 20 | H | H | H | H | H | D |
| 21 | D | D | H | H | H | H |
| 22 | D | H | D | H | H | H |
| 23 | D | H | H | D | H | H |
| 24 | D | H | H | H | D | H |
| 25 | D | H | H | H | H | D |
| 26 | H | H | D | D | H | H |
| 27 | H | H | D | H | D | H |
| 28 | H | H | D | H | H | D |
| 29 | H | H | H | D | D | H |
| 30 | H | H | H | D | H | D |
| 31 | H | H | H | H | D | D |
| 32 | D | D | D | H | H | H |
| 33 | D | D | H | D | H | H |
| 34 | D | D | H | H | D | H |
| 35 | D | D | H | H | H | D |
| 36 | D | H | D | D | H | H |
| 37 | D | H | D | H | D | H |
| 38 | D | H | D | H | H | D |
| 39 | D | H | H | D | D | H |
| 40 | D | H | H | D | H | D |
| 41 | D | H | H | H | D | D |
| 42 | H | H | D | D | D | H |
| 43 | H | H | D | D | H | D |
| 44 | H | H | D | H | D | D |
| 45 | H | H | H | D | D | D |
| 46 | D | D | D | D | H | H |
| 47 | D | D | D | H | D | H |
| 48 | D | D | D | H | H | D |
| 49 | D | D | H | D | D | H |
| 50 | D | D | H | D | H | D |
| 51 | D | D | H | H | D | D |
| 52 | D | H | D | D | D | H |
| 53 | D | H | D | D | H | D |
| 54 | D | H | D | H | D | D |
| 55 | D | H | H | D | D | D |
| 56 | H | H | D | D | D | D |
| 57 | D | D | D | D | D | H |
| 58 | D | D | D | D | H | D |
| 59 | D | D | D | H | D | D |
| 60 | D | D | H | D | D | D |
| 61 | D | H | D | D | D | D |
| 62 | D | D | D | D | D | D |

wherein Y¹, Y², Y⁴, Y⁵, Y⁶, Y⁷, Y^{10a}, Y^{10b}, Y^{11a}, Y^{11b}, Y^{12a}, Y^{12b}, Y^{13a} and Y^{13b} are all H.

7. The compound of claim 5, wherein the level of deuterium incorporation at each of Y Y^{3a}, Y^{3b}, Y^{8a}, Y^{8b}, Y^{9a} and Y^{9b} designated as deuterium is at least 52.5%, at least 75%, at least 82.5%, at least 90%, at least 95%, at least 97%, at least 98%, or at least 99%.

8. The compound of claim 1, wherein at least one of Y¹, Y², Y^{3a}, Y^{3b}, Y⁵, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} is deuterium.

9. The compound of claim 8, wherein the compound is selected from any one of the compounds set forth in the table below:
wherein Y⁴ and Y⁶ are all H.

| | Y^{10a}, | Y^{11a}, | Y^{12a}, | Y^{13a}, | Y^{3a}, | Y^{8a}, | Y^{9a}, | | |
|---|---|---|---|---|---|---|---|---|---|
| Compound | Y^{10b} | Y^{11b} | Y^{12b} | Y^{13b} | Y^{3b} | Y^{8b} | Y^{9b} | Y5 | Y7 |
| 63 | D | H | H | D | H | H | H | H | H |
| 64 | D | H | H | H | D | H | H | H | H |
| 65 | D | H | H | H | H | D | H | H | H |
| 66 | D | H | H | H | H | H | D | H | H |
| 67 | D | H | H | H | H | H | H | D | H |
| 68 | D | H | H | H | H | H | H | H | D |
| 69 | H | H | D | D | H | H | H | H | H |
| 70 | H | H | D | H | D | H | H | H | H |
| 71 | H | H | D | H | H | D | H | H | H |
| 72 | H | H | D | H | H | H | D | H | H |
| 73 | H | H | D | H | H | H | H | D | H |
| 74 | H | H | D | H | H | H | H | H | D |
| 75 | H | H | H | D | H | H | D | H | H |
| 76 | H | H | H | D | H | H | H | D | H |
| 77 | H | H | H | D | H | H | H | H | D |
| 78 | H | H | H | H | D | H | H | D | H |
| 79 | H | H | H | H | H | D | H | D | H |
| 80 | H | H | H | H | H | D | H | H | D |
| 81 | H | H | H | H | H | H | D | H | D |
| 82 | D | D | H | D | H | H | H | H | H |
| 83 | D | D | H | H | D | H | H | H | H |
| 84 | D | D | H | H | H | D | H | H | H |
| 85 | D | D | H | H | H | H | D | H | H |
| 86 | D | D | H | H | H | H | H | D | H |
| 87 | D | D | H | H | H | H | H | H | D |
| 88 | D | H | D | D | H | H | H | H | H |
| 89 | D | H | D | H | D | H | H | H | H |
| 90 | D | H | D | H | H | D | H | H | H |
| 91 | D | H | D | H | H | H | D | H | H |
| 92 | D | H | D | H | H | H | H | D | H |
| 93 | D | H | D | H | H | H | H | H | D |
| 94 | D | H | H | D | D | H | H | H | H |
| 95 | D | H | H | D | H | D | H | H | H |
| 96 | D | H | H | D | H | H | D | H | H |
| 97 | D | H | H | D | H | H | H | D | H |
| 98 | D | H | H | D | H | H | H | H | D |
| 99 | D | H | H | H | D | D | H | H | H |
| 100 | D | H | H | H | D | H | D | H | H |
| 101 | D | H | H | H | D | H | H | D | H |
| 102 | D | H | H | H | D | H | H | H | D |
| 103 | D | H | H | H | H | D | D | H | H |
| 104 | D | H | H | H | H | D | H | D | H |
| 105 | D | H | H | H | H | D | H | H | D |
| 106 | D | H | H | H | H | H | D | D | H |
| 107 | D | H | H | H | H | H | D | H | D |
| 108 | D | H | H | H | H | H | H | D | D |
| 109 | H | H | D | D | D | H | H | H | H |
| 110 | H | H | D | D | H | D | H | H | H |
| 111 | H | H | D | D | H | H | D | H | H |
| 112 | H | H | D | D | H | H | H | D | H |
| 113 | H | H | D | D | H | H | H | H | D |
| 114 | H | H | D | H | D | D | H | H | H |
| 115 | H | H | D | H | D | H | D | H | H |
| 116 | H | H | D | H | D | H | H | D | H |
| 117 | H | H | D | H | D | H | H | H | D |
| 118 | H | H | D | H | H | D | D | H | H |
| 119 | H | H | D | H | H | D | H | D | H |
| 120 | H | H | D | H | H | D | H | H | D |
| 121 | H | H | D | H | H | H | D | D | H |
| 122 | H | H | D | H | H | H | D | H | D |
| 123 | H | H | D | H | H | H | H | D | D |
| 124 | H | H | H | D | D | H | D | H | H |
| 125 | H | H | H | D | D | H | H | D | H |
| 126 | H | H | H | D | D | H | H | H | D |
| 127 | H | H | H | D | H | D | D | H | H |
| 128 | H | H | H | D | H | D | H | D | H |
| 129 | H | H | H | D | H | D | H | H | D |
| 130 | H | H | H | D | H | H | D | D | H |
| 131 | H | H | H | D | H | H | D | H | D |
| 132 | H | H | H | D | H | H | H | D | D |
| 133 | H | H | H | H | D | D | H | D | H |
| 134 | H | H | H | H | D | D | H | H | D |
| 135 | H | H | H | H | D | H | D | D | H |
| 136 | H | H | H | H | D | H | D | H | D |
| 137 | H | H | H | H | D | H | H | D | D |
| 138 | H | H | H | H | H | D | D | D | H |
| 139 | H | H | H | H | H | D | D | H | D |
| 140 | H | H | H | H | H | D | H | D | D |
| 141 | H | H | H | H | H | H | D | D | D |
| 142 | D | D | D | H | D | H | H | H | H |
| 143 | D | D | D | H | H | D | H | H | H |
| 144 | D | D | D | H | H | H | D | H | H |
| 145 | D | D | D | H | H | H | H | D | H |
| 146 | D | D | D | H | H | H | H | H | D |
| 147 | D | D | H | D | D | H | H | H | H |
| 148 | D | D | H | D | H | D | H | H | H |
| 149 | D | D | H | D | H | H | D | H | H |
| 150 | D | D | H | D | H | H | H | D | H |
| 151 | D | D | H | D | H | H | H | H | D |
| 152 | D | D | H | H | D | D | H | H | H |
| 153 | D | D | H | H | D | H | D | H | H |
| 154 | D | D | H | H | D | H | H | D | H |
| 155 | D | D | H | H | D | H | H | H | D |
| 156 | D | D | H | H | H | D | D | H | H |
| 157 | D | D | H | H | H | D | H | D | H |
| 158 | D | D | H | H | H | D | H | H | D |
| 159 | D | D | H | H | H | H | D | D | H |
| 160 | D | D | H | H | H | H | D | H | D |
| 161 | D | D | H | H | H | H | H | D | D |
| 162 | D | H | D | D | D | H | H | H | H |
| 163 | D | H | D | D | H | D | H | H | H |
| 164 | D | H | D | D | H | H | D | H | H |
| 165 | D | H | D | D | H | H | H | D | H |
| 166 | D | H | D | D | H | H | H | H | D |
| 167 | D | H | D | H | D | D | H | H | H |
| 168 | D | H | D | H | D | H | D | H | H |
| 169 | D | H | D | H | D | H | H | D | H |
| 170 | D | H | D | H | D | H | H | H | D |
| 171 | D | H | D | H | H | D | D | H | H |
| 172 | D | H | D | H | H | D | H | D | H |
| 173 | D | H | D | H | H | D | H | H | D |
| 174 | D | H | D | H | H | H | D | D | H |
| 175 | D | H | D | H | H | H | D | H | D |
| 176 | D | H | D | H | H | H | H | D | D |
| 177 | D | H | H | D | D | D | H | H | H |
| 178 | D | H | H | D | D | H | D | H | H |
| 179 | D | H | H | D | D | H | H | D | H |
| 180 | D | H | H | D | D | H | H | H | D |
| 181 | D | H | H | D | H | D | D | H | H |
| 182 | D | H | H | D | H | D | H | D | H |
| 183 | D | H | H | D | H | D | H | H | D |
| 184 | D | H | H | D | H | H | D | D | H |
| 185 | D | H | H | D | H | H | D | H | D |
| 186 | D | H | H | D | H | H | H | D | D |
| 187 | D | H | H | H | D | D | D | H | H |
| 188 | D | H | H | H | D | D | H | D | H |
| 189 | D | H | H | H | D | D | H | H | D |
| 190 | D | H | H | H | D | H | D | D | H |
| 191 | D | H | H | H | D | H | D | H | D |
| 192 | D | H | H | H | D | H | H | D | D |
| 193 | D | H | H | H | H | D | D | D | H |
| 194 | D | H | H | H | H | D | D | H | D |
| 195 | D | H | H | H | H | D | H | D | D |
| 196 | D | H | H | H | H | H | D | D | D |
| 197 | H | H | D | D | D | D | H | H | H |
| 198 | H | H | D | D | D | H | D | H | H |
| 199 | H | H | D | D | D | H | H | D | H |
| 200 | H | H | D | D | D | H | H | H | D |
| 201 | H | H | D | D | H | D | D | H | H |
| 202 | H | H | D | D | H | D | H | D | H |
| 203 | H | H | D | D | H | D | H | H | D |
| 204 | H | H | D | D | H | H | D | D | H |
| 205 | H | H | D | D | H | H | D | H | D |
| 206 | H | H | D | D | H | H | H | D | D |
| 207 | H | H | D | H | D | D | D | H | H |
| 208 | H | H | D | H | D | D | H | D | H |
| 209 | H | H | D | H | D | D | H | H | D |
| 210 | H | H | D | H | D | H | D | D | H |
| 211 | H | H | D | H | D | H | D | H | D |
| 212 | H | H | D | H | D | H | H | D | D |
| 213 | H | H | D | H | H | D | D | D | H |
| 214 | H | H | D | H | H | D | D | H | D |
| 215 | H | H | D | H | H | D | H | D | D |
| 216 | H | H | D | H | H | H | D | D | D |
| 217 | H | H | H | D | D | D | D | H | H |
| 218 | H | H | H | D | D | D | H | D | H |
| 219 | H | H | H | D | D | D | H | H | D |
| 220 | H | H | H | D | D | H | D | D | H |
| 221 | H | H | H | D | D | H | D | H | D |
| 222 | H | H | H | D | D | H | H | D | D |
| 223 | H | H | H | D | H | D | D | D | H |
| 224 | H | H | H | D | H | D | D | H | D |
| 225 | H | H | H | D | H | D | H | D | D |
| 226 | H | H | H | D | H | H | D | D | D |
| 227 | H | H | H | H | D | D | D | D | H |
| 228 | H | H | H | H | D | D | D | H | D |
| 229 | H | H | H | H | D | D | H | D | D |
| 230 | H | H | H | H | D | H | D | D | D |
| 231 | H | H | H | H | H | D | D | D | D |
| 232 | D | D | D | D | D | H | H | H | H |
| 233 | D | D | D | D | H | D | H | H | H |
| 234 | D | D | D | D | H | H | D | H | H |
| 235 | D | D | D | D | H | H | H | D | H |
| 236 | D | D | D | D | H | H | H | H | D |
| 237 | D | D | D | H | D | D | H | H | H |
| 238 | D | D | D | H | D | H | D | H | H |
| 239 | D | D | D | H | D | H | H | D | H |
| 240 | D | D | D | H | D | H | H | H | D |
| 241 | D | D | D | H | H | D | D | H | H |
| 242 | D | D | D | H | H | D | H | D | H |
| 243 | D | D | D | H | H | D | H | H | D |
| 244 | D | D | D | H | H | H | D | D | H |
| 245 | D | D | D | H | H | H | D | H | D |
| 246 | D | D | D | H | H | H | H | D | D |
| 247 | D | D | H | D | D | D | H | H | H |
| 248 | D | D | H | D | D | H | D | H | H |
| 249 | D | D | H | D | D | H | H | D | H |
| 250 | D | D | H | D | D | H | H | H | D |
| 251 | D | D | H | D | H | D | D | H | H |
| 252 | D | D | H | D | H | D | H | D | H |
| 253 | D | D | H | D | H | D | H | H | D |
| 254 | D | D | H | D | H | H | D | D | H |
| 255 | D | D | H | D | H | H | D | H | D |
| 256 | D | D | H | D | H | H | H | D | D |
| 257 | D | D | H | H | D | D | D | H | H |
| 258 | D | D | H | H | D | D | H | D | H |
| 259 | D | D | H | H | D | D | H | H | D |
| 260 | D | D | H | H | D | H | D | D | H |
| 261 | D | D | H | H | D | H | D | H | D |
| 262 | D | D | H | H | D | H | H | D | D |
| 263 | D | D | H | H | H | D | D | D | H |
| 264 | D | D | H | H | H | D | D | H | D |
| 265 | D | D | H | H | H | D | H | D | D |
| 266 | D | D | H | H | H | H | D | D | D |
| 267 | D | H | D | D | D | D | H | H | H |
| 268 | D | H | D | D | D | H | D | H | H |
| 269 | D | H | D | D | D | H | H | D | H |
| 270 | D | H | D | D | D | H | H | H | D |
| 271 | D | H | D | D | H | D | D | H | H |
| 272 | D | H | D | D | H | D | H | D | H |
| 273 | D | H | D | D | H | D | H | H | D |
| 274 | D | H | D | D | H | H | D | D | H |
| 275 | D | H | D | D | H | H | D | H | D |
| 276 | D | H | D | D | H | H | H | D | D |
| 277 | D | H | D | H | D | D | D | H | H |
| 278 | D | H | D | H | D | D | H | D | H |
| 279 | D | H | D | H | D | D | H | H | D |
| 280 | D | H | D | H | D | H | D | D | H |
| 281 | D | H | D | H | D | H | D | H | D |
| 282 | D | H | D | H | D | H | H | D | D |
| 283 | D | H | D | H | H | D | D | D | H |
| 284 | D | H | D | H | H | D | D | H | D |
| 285 | D | H | D | H | H | D | H | D | D |
| 286 | D | H | D | H | H | H | D | D | D |
| 287 | D | H | H | D | D | D | D | H | H |
| 288 | D | H | H | D | D | D | H | D | H |
| 289 | D | H | H | D | D | D | H | H | D |
| 290 | D | H | H | D | D | H | D | D | H |
| 291 | D | H | H | D | D | H | D | H | D |
| 292 | D | H | H | D | D | H | H | D | D |
| 293 | D | H | H | D | H | D | D | D | H |
| 294 | D | H | H | D | H | D | D | H | D |
| 295 | D | H | H | D | H | D | H | D | D |
| 296 | D | H | H | D | H | H | D | D | D |
| 297 | D | H | H | H | D | D | D | D | H |
| 298 | D | H | H | H | D | D | D | H | D |
| 299 | D | H | H | H | D | D | H | D | D |
| 300 | D | H | H | H | D | H | D | D | D |
| 301 | D | H | H | H | H | D | D | D | D |
| 302 | H | H | D | D | D | D | D | H | H |
| 303 | H | H | D | D | D | D | H | D | H |
| 304 | H | H | D | D | D | D | H | H | D |
| 305 | H | H | D | D | D | H | D | D | H |
| 306 | H | H | D | D | D | H | D | H | D |
| 307 | H | H | D | D | D | H | H | D | D |
| 308 | H | H | D | D | H | D | D | D | H |
| 309 | H | H | D | D | H | D | D | H | D |
| 310 | H | H | D | D | H | D | H | D | D |
| 311 | H | H | D | D | H | H | D | D | D |
| 312 | H | H | D | H | D | D | D | D | H |
| 313 | H | H | D | H | D | D | D | H | D |
| 314 | H | H | D | H | D | D | H | D | D |
| 315 | H | H | D | H | D | H | D | D | D |
| 316 | H | H | D | H | H | D | D | D | D |
| 317 | H | H | H | D | D | D | D | D | H |
| 318 | H | H | H | D | D | D | D | H | D |
| 319 | H | H | H | D | D | D | H | D | D |
| 320 | H | H | H | D | D | H | D | D | D |
| 321 | H | H | H | D | H | D | D | D | D |
| 322 | H | H | H | H | D | D | D | D | D |
| 323 | D | D | D | D | D | D | H | H | H |
| 324 | D | D | D | D | D | H | D | H | H |
| 325 | D | D | D | D | D | H | H | D | H |
| 326 | D | D | D | D | D | H | H | H | D |
| 327 | D | D | D | D | H | D | D | H | H |
| 328 | D | D | D | D | H | D | H | D | H |
| 329 | D | D | D | D | H | D | H | H | D |
| 330 | D | D | D | D | H | H | D | D | H |
| 331 | D | D | D | D | H | H | D | H | D |
| 332 | D | D | D | D | H | H | H | D | D |
| 333 | D | D | D | H | D | D | D | H | H |
| 334 | D | D | D | H | D | D | H | D | H |
| 335 | D | D | D | H | D | D | H | H | D |
| 336 | D | D | D | H | D | H | D | D | H |
| 337 | D | D | D | H | D | H | D | H | D |
| 338 | D | D | D | H | D | H | H | D | D |
| 339 | D | D | D | H | H | D | D | D | H |
| 340 | D | D | D | H | H | D | D | H | D |
| 341 | D | D | D | H | H | D | H | D | D |
| 342 | D | D | D | H | H | H | D | D | D |
| 343 | D | D | H | D | D | D | D | H | H |
| 344 | D | D | H | D | D | D | H | D | H |
| 345 | D | D | H | D | D | D | H | H | D |
| 346 | D | D | H | D | D | H | D | D | H |
| 347 | D | D | H | D | D | H | D | H | D |
| 348 | D | D | H | D | D | H | H | D | D |
| 349 | D | D | H | D | H | D | D | D | H |
| 350 | D | D | H | D | H | D | D | H | D |
| 351 | D | D | H | D | H | D | H | D | D |
| 352 | D | D | H | D | H | H | D | D | D |
| 353 | D | D | H | H | D | D | D | D | H |
| 354 | D | D | H | H | D | D | D | H | D |
| 355 | D | D | H | H | D | D | H | D | D |
| 356 | D | D | H | H | D | H | D | D | D |
| 357 | D | D | H | H | H | D | D | D | D |
| 358 | D | H | D | D | D | D | D | H | H |
| 359 | D | H | D | D | D | D | H | D | H |
| 360 | D | H | D | D | D | D | H | H | D |
| 361 | D | H | D | D | D | H | D | D | H |
| 362 | D | H | D | D | D | H | D | H | D |
| 363 | D | H | D | D | D | H | H | D | D |
| 364 | D | H | D | D | H | D | D | D | H |
| 365 | D | H | D | D | H | D | D | H | D |
| 366 | D | H | D | D | H | D | H | D | D |
| 367 | D | H | D | D | H | H | D | D | D |
| 368 | D | H | D | H | D | D | D | D | H |
| 369 | D | H | D | H | D | D | D | H | D |
| 370 | D | H | D | H | D | D | H | D | D |
| 371 | D | H | D | H | D | H | D | D | D |
| 372 | D | H | D | H | H | D | D | D | D |
| 373 | D | H | H | D | D | D | D | D | H |
| 374 | D | H | H | D | D | D | D | H | D |
| 375 | D | H | H | D | D | D | H | D | D |
| 376 | D | H | H | D | D | H | D | D | D |
| 377 | D | H | H | D | H | D | D | D | D |
| 378 | D | H | H | H | D | D | D | D | D |
| 379 | H | H | D | D | D | D | D | D | H |
| 380 | H | H | D | D | D | D | D | H | D |
| 381 | H | H | D | D | D | D | H | D | D |
| 382 | H | H | D | D | D | H | D | D | D |
| 383 | H | H | D | D | H | D | D | D | D |
| 384 | H | H | D | H | D | D | D | D | D |
| 385 | H | H | H | D | D | D | D | D | D |
| 386 | D | D | D | D | D | D | D | H | H |
| 387 | D | D | D | D | D | D | H | D | H |
| 388 | D | D | D | D | D | D | H | H | D |
| 389 | D | D | D | D | D | H | D | D | H |
| 390 | D | D | D | D | D | H | D | H | D |
| 391 | D | D | D | D | D | H | H | D | D |
| 392 | D | D | D | D | H | D | D | D | H |
| 393 | D | D | D | D | H | D | D | H | D |
| 394 | D | D | D | D | H | D | H | D | D |
| 395 | D | D | D | D | H | H | D | D | D |
| 396 | D | D | D | H | D | D | D | D | H |
| 397 | D | D | D | H | D | D | D | H | D |
| 398 | D | D | D | H | D | D | H | D | D |
| 399 | D | D | D | H | D | H | D | D | D |
| 400 | D | D | D | H | H | D | D | D | D |
| 401 | D | D | H | D | D | D | D | D | H |
| 402 | D | D | H | D | D | D | D | H | D |
| 403 | D | D | H | D | D | D | H | D | D |
| 404 | D | D | H | D | D | H | D | D | D |
| 405 | D | D | H | D | H | D | D | D | D |
| 406 | D | D | H | H | D | D | D | D | D |
| 407 | D | H | D | D | D | D | D | D | H |
| 408 | D | H | D | D | D | D | D | H | D |
| 409 | D | H | D | D | D | D | H | D | D |
| 410 | D | H | D | D | D | H | D | D | D |
| 411 | D | H | D | D | H | D | D | D | D |
| 412 | D | H | D | H | D | D | D | D | D |
| 413 | D | H | H | D | D | D | D | D | D |
| 414 | H | H | D | D | D | D | D | D | D |
| 415 | D | D | D | D | D | D | D | D | H |
| 416 | D | D | D | D | D | D | D | H | D |
| 417 | D | D | D | D | D | D | H | D | D |
| 418 | D | D | D | D | D | H | D | D | D |
| 419 | D | D | D | D | H | D | D | D | D |
| 420 | D | D | D | H | D | D | D | D | D |
| 421 | D | D | H | D | D | D | D | D | D |
| 422 | D | H | D | D | D | D | D | D | D |
| 423 | D | D | D | D | D | D | D | D | D |
| 424 | H | D | D | H | H | H | H | H | H |
| 425 | D | D | H | H | H | H | H | H | H |

10. The compound of claim 8, wherein the level of deuterium incorporation at each of Y¹, Y², Y^{3a}, Y^{3b}, Y⁵, Y⁷, Y^{8a}, Y^{8b}, Y^{9a}, Y^{9b}, Y^{10a}, Y^{10b}, Y^{11a}, Y^{11b}, Y^{12a}, Y^{12b}, Y^{13a} and Y^{13b} designated as deuterium is at least 52.5%, at least 75%, at least 82.5%, at least 90%, at least 95%, at least 97%, at least 98%, or at least 99%.

11. The compound of claim 1, wherein the compound is selected from the group consisting of: 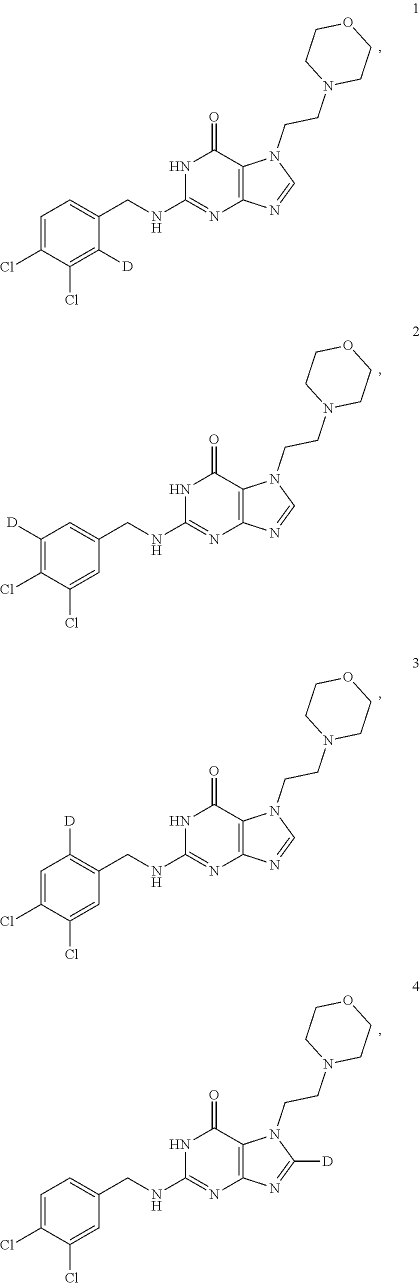 [SMILES: [2H]c1c(CNc2nc3ncn(CCN4CCOCC4)c3c(=O)[nH]2)ccc(Cl)c1Cl ; [2H]c1cc(CNc2nc3ncn(CCN4CCOCC4)c3c(=O)[nH]2)cc(Cl)c1Cl ; [2H]c1cc(Cl)c(Cl)cc1CNc1nc2ncn(CCN3CCOCC3)c2c(=O)[nH]1 ; [2H]c1nc2nc(NCc3ccc(Cl)c(Cl)c3)[nH]c(=O)c2n1CCN1CCOCC1] 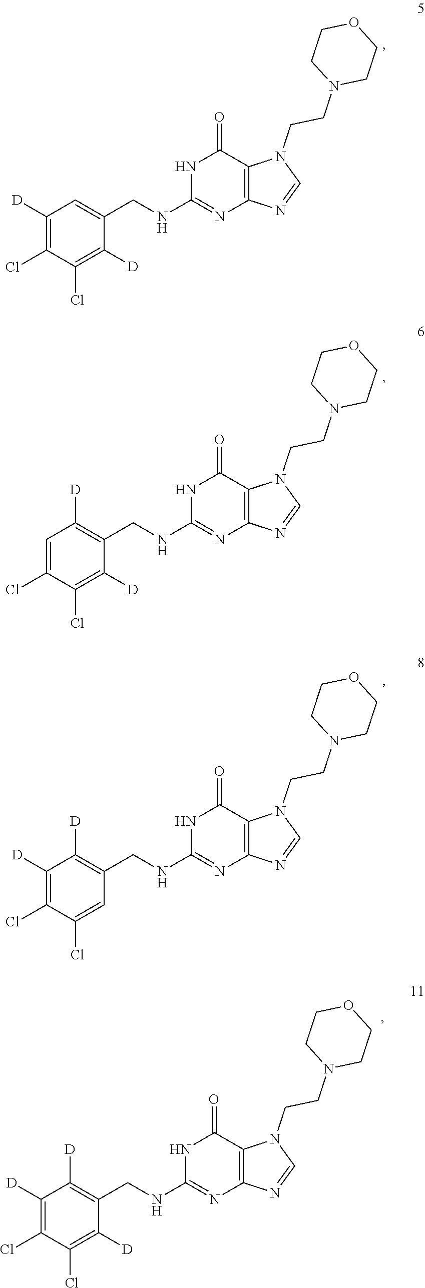 [SMILES: [2H]c1cc(CNc2nc3ncn(CCN4CCOCC4)c3c(=O)[nH]2)c([2H])c(Cl)c1Cl ; [2H]c1cc(Cl)c(Cl)c([2H])c1CNc1nc2ncn(CCN3CCOCC3)c2c(=O)[nH]1 ; [2H]c1c(CNc2nc3ncn(CCN4CCOCC4)c3c(=O)[nH]2)cc(Cl)c(Cl)c1[2H] ; [2H]c1c([2H])c(CNc2nc3ncn(CCN4CCOCC4)c3c(=O)[nH]2)c([2H])c(Cl)c1Cl] 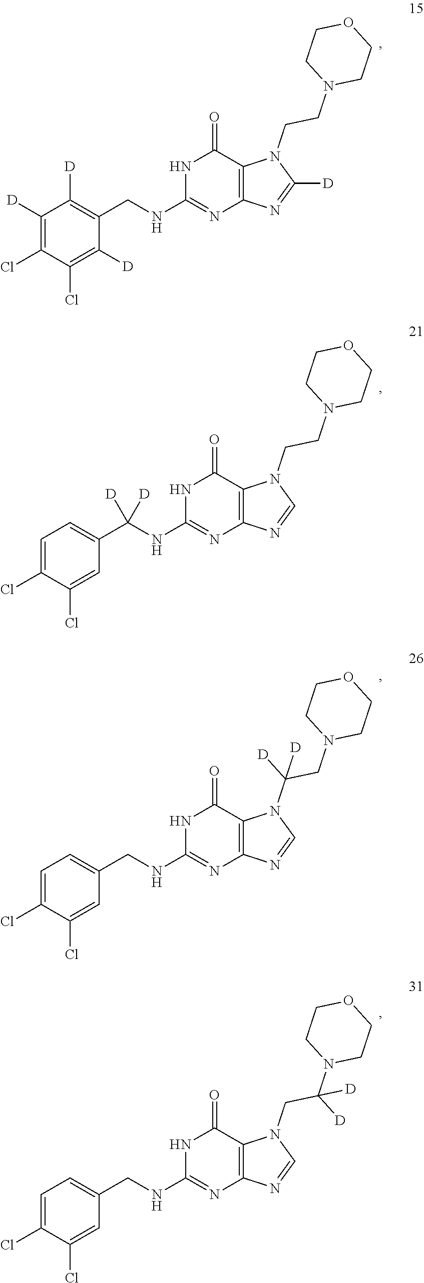 [SMILES: [2H]c1c([2H])c(CNc2nc3nc([2H])n(CCN4CCOCC4)c3c(=O)[nH]2)c([2H])c(Cl)c1Cl ; [2H]C([2H])(Nc1nc2ncn(CCN3CCOCC3)c2c(=O)[nH]1)c1ccc(Cl)c(Cl)c1 ; [2H]C([2H])(CN1CCOCC1)n1cnc2nc(NCc3ccc(Cl)c(Cl)c3)[nH]c(=O)c21 ; [2H]C([2H])(Cn1cnc2nc(NCc3ccc(Cl)c(Cl)c3)[nH]c(=O)c21)N1CCOCC1] 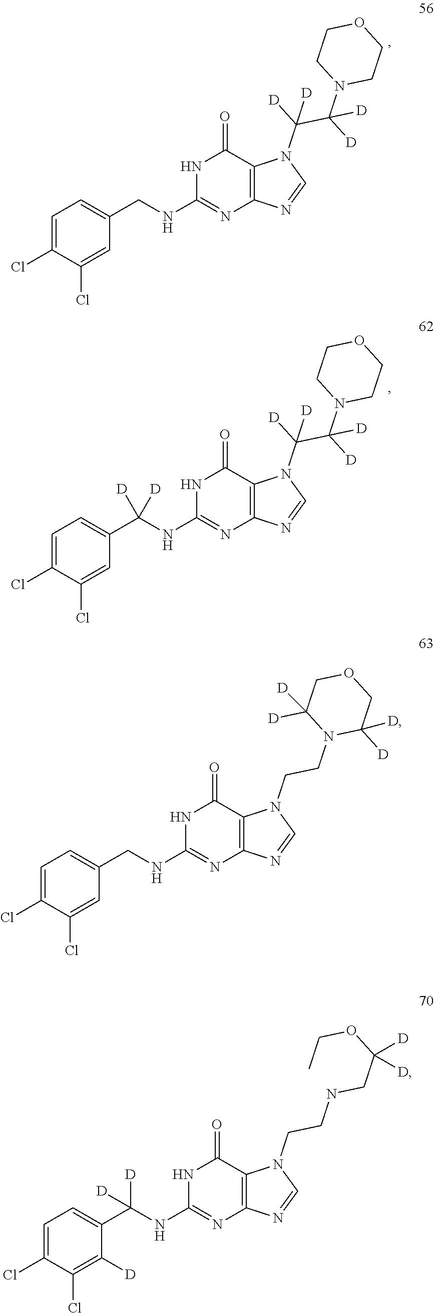 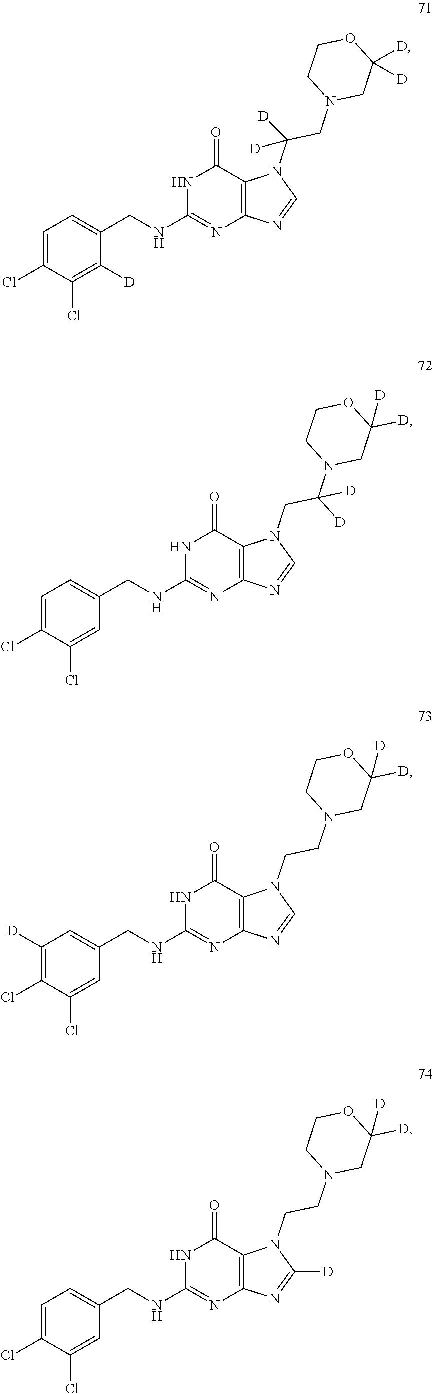 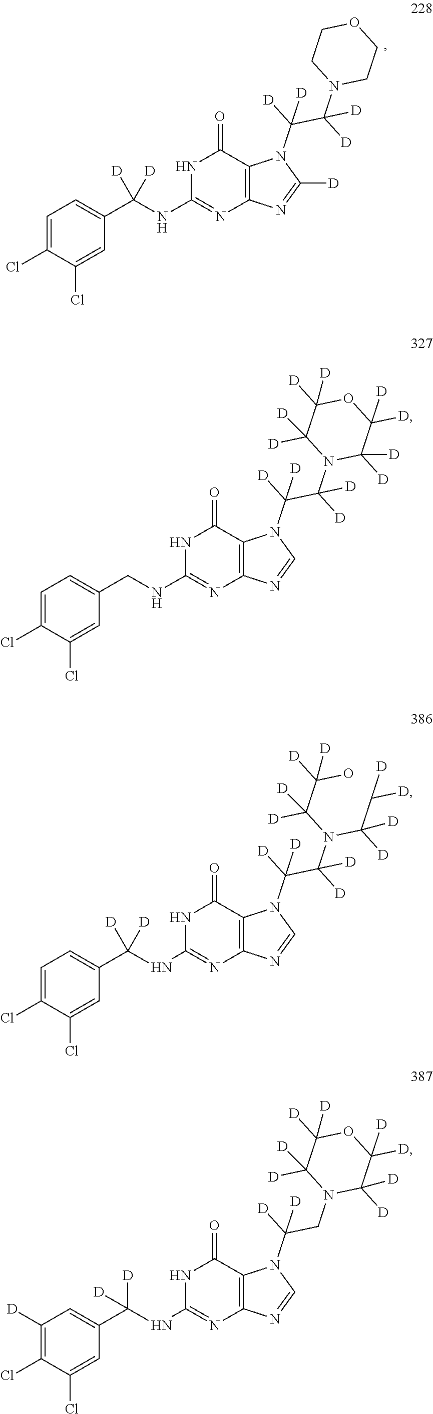 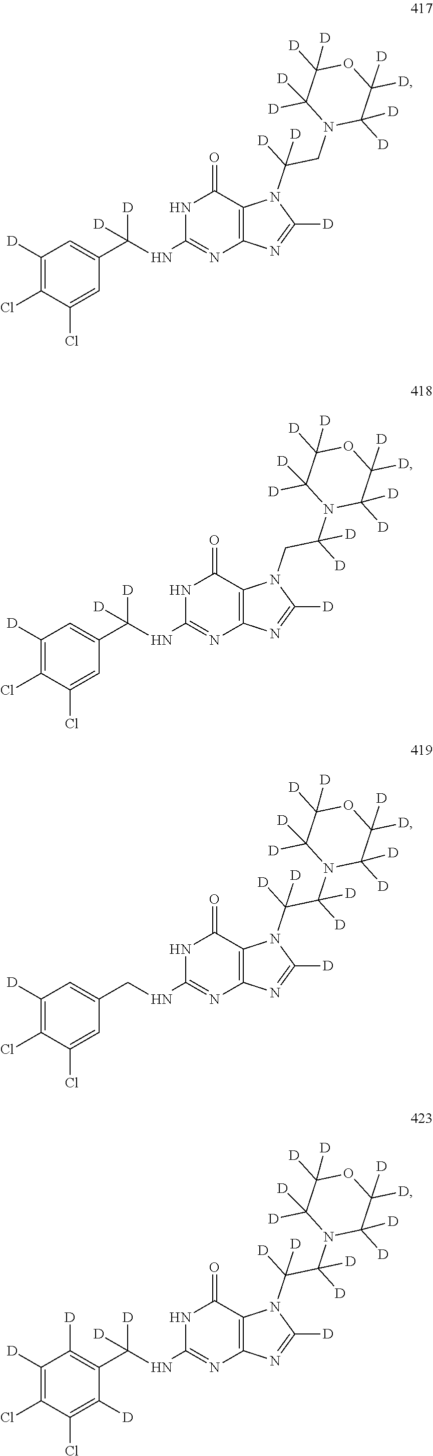 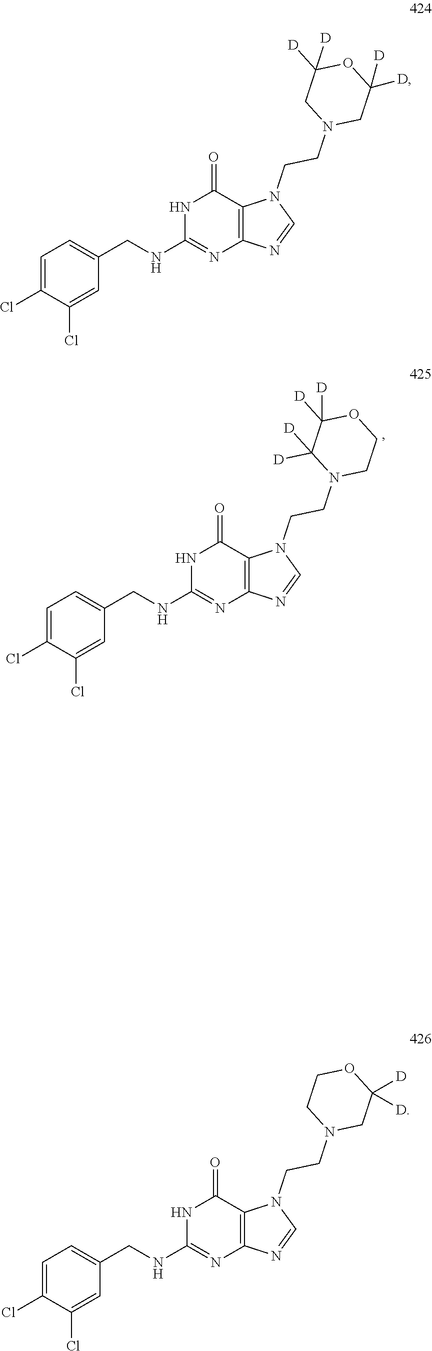
and pharmaceutically acceptable salts thereof.

12. A pharmaceutical composition comprising a compound of claim 1, and a pharmaceutically acceptable excipient.

13. A method of inhibiting growth of Clostridium difficile in vitro, the method comprising contacting, in an appropriate culture medium in the absence of oxygen, Clostridium difficile with an effective amount of a compound of claim 1.

14. A method of inhibiting growth of Clostridium difficile in vitro, the method comprising contacting, in an appropriate culture medium in the absence of oxygen, Clostridium difficile with an effective amount of a pharmaceutical composition of claim 12.
